(19) United States
(12) Patent Application Publication
(10) Pub. No.: US 2009/0104124 A1
(43) Pub. Date: Apr. 23, 2009

(54) Paramagnetic Complexes with Pendant Crown Compounds Showing Improved Targeting- Specificity as MRI Contrast Agents

(76) Inventors: Wing-Tak Wong, Hong Kong (HK); Cong Li, Changsha (CN)

Correspondence Address: COOPER & DUNHAM, LLP, 30 Rockefeller Plaza, 20th Floor, NEW YORK, NY, 10112, US

(21) Appl. No.: 11/873,832

(22) Filed: Oct. 17, 2007

(51) Int. Cl. A61K 49/12

(52) U.S. Cl. 424/9.32

(57) ABSTRACT

Potential magnetic resonance imaging (MRI) contrast agents that are functionalized with crown compounds have been synthesized, and show target-specificity to the kidney and liver, along with excellent water solubility, high MR intensity enhancement efficiency, long resident lifetime, low dosage requirement, low cytotoxicity and prolonged excretion rate.

### BACKGROUND OF THE INVENTION

Magnetic resonance imaging (MRI) as a non-invasive diagnostic technology is not only widely used to reveal anatomical structures and detect lesions in vivo, but also plays a very promising role in the future characterization and measurement of biologic process at the cellular and molecular level. This is due to the fact that it has fewer limitations on the observation depth and spatial resolution compared with other diagnostic techniques such as bio-luminescent imaging or positron emission tomography (PET). The current technology relies on the detection of the energy emitted when the hydrogen nuclei in the water that is contained in tissues and body fluids return to a ground state subsequent to excitation with a radio frequency. However, because the relaxation rate of the relevant hydrogen nuclei is too slow to generate detectable amounts of energy, molecular probe detection by MRI is several orders of magnitude less sensitive than PET or other nuclear technology. Therefore, much effort has been made to develop novel MRI contrast agents (CAs) that act as catalysts to accelerate the relaxation rates of water proton nuclei in their surroundings, to enhance the image contrast between normal and diseased tissues and organs. Currently, more than 35% of MR examinations are performed after the administration of a contrast agent.

Signal intensity in biological MRI depends largely on the value of the longitudinal relaxation rate (1/T₁), and the transverse relaxation rate (1/T₂) of water protons. Contrast agents will increase 1/T₁ or 1/T₂, depending on the nature of the agent and the strength of the magnetic field, MRI contrast agents (CAs) include in T₁-weighted or T₂-weighted agents according to whether the pulse sequences emphasize the changes in 1/T₂ or 1/T₂. Among many paramagnetic metal ions, gadolinium (III) is very efficient in enhancing T₁-weighted proton relaxation rates by virtue of its seven unpaired electrons and its relatively slow electronic relaxation rate induced by its symmetric electronic S-state. However, the free Gd³⁺ ion is very toxic due to its disruption of the critical Ca²⁺ required in the signaling process. Thus, suitable ligand systems are required to chelate the Gd³⁺ ion strongly before its excretion from the body. Like other pharmaceuticals, MRI CAs must also be biocompatible and satisfy basic features such as water solubility, thermodynamic stabilities, kinetic inertness, and suitable excretion rates.

The efficiency of a given MRI contrast agent in catalyzing the rate of relaxation of the water protons can be most conveniently expressed by the equations 1, 2, and 3, in which the rate of relaxation of the water proton signal R₁, is primarily determined by the paramagnetic contribution R₁ₚ, rather than the diamagnetic term R_{1d}. The paramagnetic term is made up, in turn, of outer sphere (os), second (ss) and inner sphere (is) contributions.

$$\begin{matrix}{R}_{1} = {R}_{1 p} + {R}_{1 d} & Equation 1 \\ {R}_{1 p} = {R}_{1 p}^{IS} + {R}_{1 p}^{SS} + {R}_{1 p}^{OS} & Equation 2 \\ {R}_{1 p} = \frac{[ M ] q}{55.6} \frac{1}{{T}_{1 M} + {τ}_{M}} & Equation 3\end{matrix}$$

The paramagnetic contribution to the relaxation rate ascribed to inner sphere waters (equation 3) is governed by the relative importance of T_{1M}, the longitudinal bound water proton relaxation time, and τₘ, the water proton exchange lifetime. The paramagnetic relaxation rate per unit of complex concentration, is defined as the relaxivity of the complex, r₁ₚ, and is directly proportional to the number of coordinated water molecules, q. The water proton longitudinal relaxation rate (1/T_{1M}) can be given by the modified Solomon-Bloembergen equations, (Bloembergen, J. Chem. Phys. 1957, 27, 572; Solomon et al., J. Chem. Phys., 1956, 25, 261), as in Equations 4-6.

$$\begin{matrix}\frac{1}{{T}_{1 M}} = \frac{1}{{T}_{1 M}^{DD}} + \frac{1}{{T}_{1 M}^{SC}} & Equation 4 \\ \frac{1}{{T}_{1 M}^{DD}} = \frac{2}{15} \frac{{γ}_{11}^{2} {g}_{e}^{2} {μ}_{B}^{2} S ( S + 1 )}{{r}_{H}^{6}} {( \frac{{μ}_{o}}{4 π} )}^{2} [ \frac{3 {τ}_{e 1}}{1 + {ω}_{H}^{2} {τ}_{e 1}^{2}} + \frac{7 {τ }_{e 2}}{1 + {ω}_{S}^{2} {τ}_{e 2}^{2}} ] & Equation 5 \\ \frac{1}{{T}_{1 M}^{SC}} = \frac{2 S ( S + 1 )}{3} {( \frac{A}{h} )}^{2} ( \frac{{τ}_{e 2}}{1 + {ω}_{S}^{2} {τ}_{e 2}^{2}} ) & Equation 6\end{matrix}$$

where

$$\frac{1}{{T}_{1 M}^{DD}}$$

is the water proton longitudinal relaxation rate due to dipolar contribution,

$$\frac{1}{{T}_{1 M}^{SC}}$$

is the rate that is due to scalar contribution, S is the electron spin quantum number (7/2 for Gd³⁺), γ_{H} is the proton nuclear magnetogyric ration, μ_{B} is the Bohrmagneton, gₑ is the Landè factor for the free electron, μ₀/4π is the magnetic permeability in a vacuum, r_{H} is the distance between the metal ion and the bound water protons, and ω_{H} and ω_{S} are the proton and electron Larmor frequencies, respectively. A/η is the hyperfine or scalar coupling constant between the electron of the paramagnetic centre and the proton of the coordinated water. The correlation times (T_{ci} and Tₑᵢ) that are characteristic of the relaxation process are depicted as Equations 7-8.

$$\begin{matrix}\frac{1}{{τ}_{ci}} = \frac{1}{{τ}_{R}} + \frac{1}{{T}_{iE}} + \frac{1}{{τ}_{M}} \\ where i = 1 , 2 & Equation 7 \\ \frac{1}{{τ}_{ei}} = \frac{1}{{T}_{iE}} + \frac{1}{{τ}_{M}} \\ where i = 1 , 2 & Equation 8\end{matrix}$$

where τ_{R} is the reorientational correlation time of the metal-proton vector, and T_{iE} is the electron spin relaxation time of the metal ion.

It is obvious from the aforementioned equations that the proton relaxivity is influenced by numerous parameters. According to Equation 3, the relaxivity is linearly proportional to the number of directly coordinated water molecules q. However, when the bound water proton relaxation is very fast (T_{1M}<<τ_{M}), the water exchange rate is the only determining factor for relaxivity. However, if the proton exchange rate is fast enough (T_{M}>>τ_{M}), then the observed proton relaxivity is exclusively determined by the water proton relaxation time, T_{1M}, which itself depends on the rate of proton exchange, rotation and electronic relaxation (Equations 4-8). Moreover, the Gd-proton distance (r_{H}, see FIG. 1) influences the relaxivity in an obvious manner because the slightly greater length of this distance results in a significant decrease in relaxivity due to the sixth-power dependence.

The relaxivities of the Gd-based contrast agents that are currently in clinical use are much lower than the theoretically attainable value (>100 mM⁻¹s⁻¹). The reason for this is mainly that neither their proton exchange rate (kₑₓ or 1/τ_{M}), nor their rotational correlation time (τ_{R}) is optical. The resident lifetime of inner-sphere water molecule τ_{M} plays a dual role in the determination of the relaxivity of the complexes. It not only modulates the efficiency of the relaxivity exchange from the inner-sphere of the metal to the bulk (Equation 3), but also contributes to the overall correlation time, τ_{c}, that governs the dipole-dipole interaction between the electron and the nuclear spin (Equations 5-7). To increase the relaxivity, the optimal τ_{M} should be in the range of 10-30 ns in theory, with a corresponding kₑₓ in the right order of magnitude of 10⁷-10⁹ s⁻¹, depending on factors such as the magnetic field strength, electron spin relaxation rate, and the molecular tumbling time.

For the contrast agents in clinical use, the relaxivity of these Gd³⁺ complexes with a small molecular weight is limited by the rotation correlation time, τ_{R}. From the Equations 3, 5, and 7, two routes of obtaining long τ_{R} values are as fellows. The first is the formation of covalent conjugates between the paramagnetic complex and slowly tumbling substrates such as polylysine (Aime et al., Chem. Comm., 1999, 1577), dextran (Corsi et al., Chem. Eur. J., 2001, 7, 64), and dendrimers (Stiriba, et al., Angew. Chem. Int. Ed., 2002, 41, 1329). The second is the composition of host-guest non-covalent interactions between suitably functionalized complexes and slowly tumbling substrates such as human serum albumin (HSA) (Aime et al., J. Biol. Inorg. Chem., 2000, 5, 488). The large adducts result in a slowing down of the molecular motion, thus allowing τ_{R} to reach values in the range of tenths of a nanosecond. It is worth mentioning that the second method of lengthening τ_{R} by the specific binding between the functionalized small molecular paramagnetic probe and the large adducts not only yields a much higher relaxivity than that of the free complex, but also effectively decreases the dosage of contrast agent that needs to be administered.

Over the past two decades, a large number of Gd³⁺ complexes that are based on acyclic polyaminocarboxylic chelates and polyaza macrocycles have been synthesized. Polyaminocarboxylic ligands provide nitrogen and oxygen atoms as donors, which are hard enough to stabilize the Ln³⁺ ions by forming strong Ln—O and Ln—N bonds.

Furthermore, the presence of acetates or other pendant arms such as amides keeps the whole complex more hydrophilic and soluble in water, which is an essential criterion for a potential MRI contrast agent. Among the seven clinically approved gadolinium(III) chelates, [Gd(DTPA)(H₂O)² (Magnevist™), [Gd(DTPA-BMA)(H₂O)] (Omniscan™), [Gd(BOPTA)(H₂O)]²⁻ (MultiHance™), and [Gd(DTPA-BMEA)(H₂O)] (OptiMARK™ ) are complexes of acyclic ligands, and the remaining three are chelated by macrocycles (FIG. 2). As the first approved MRI contrast agent, [Gd(DTPA)(H₂O)]²⁻ (Magnevist™) forms a very stable complex with Gd³⁺ (log K 24.6, where K is the formation constant of the metal complex), and shows other advantages such as low toxicity, a low incidence of adverse reactions, and rapid clearance from the body. Gd-DTPA is usually administered at a dosage of 0.1 mmol/kg of body weight. After injection, the complex diffuses from the blood into the extracellular space, and quickly excretes from the human body via glomular filtration (renal clearance). Despite the high in vitro thermodynamic stability constant, Gd-DTPA and similar acyclic lanthanide complexes are relatively kinetically labile, and undergo metal exchange via different pathways. For example, DTPA shows poor selectivity for Gd³⁺ over Zn²⁺, which results in a higher concentration of Gd³⁺ release in vivo than predicted. Moreover, Gd-DTPA is excreted rapidly from the body with poor selectivity and tissue-specificity. In addition, the complex is a salt under physiological conditions, and the cationic counterions lead to a high osmolality (1960 mOsmol/kg for Gd-DTPA; 290 mOsmol/kg for body fluid). This large osmolality difference between the complex solution and the body fluid causes pain and tissue sloughing when extravasated upon injection.

Compared to the kinetic lability of acyclic lanthanide chelates that suffer transmetallation with consequent increase of toxicity in vivo, polyaza macrocyclic ligands are particularly well suited to form kinetically inert complexes. 1,4,7,10-tetra(carboxymethyl)-1,4,7,10-tetraazacyclododecane (DOTA), a twelve-membered tetraazamacrocycle ring (cyclen) bearing four carboxymethyl (acetate) pendant groups, is found to be one of the strongest chelators for Gd³⁺, at about three orders of efficiency in complexation higher than DTPA. Its kinetic inertness stems from the encapsulating nature of the macrocyclic ligands (macrocyclic effect), and their thermodynamic stabilization due to the large degree of preorganization of the ligand. Hence, it is thus no surprise that DOTA has become the parent molecule of an ever-growing family of macrocyclic Ln³⁺ chelating agents. Even though Gd-DOTA has only one negative charge, its osmolality is still high (1400 mOsmol/kg). To reduce it, neutral [Gd(HP-DO3A)(H₂O)] (ProHance™) and [Gd(DO3A-butrol)(H₂O)] (Gadovist™) were developed. It should be emphasized that the relaxivities of these three Gd³⁺ complexes are still high enough for application {Gd-DOTA: 3.4 mM⁻¹s⁻¹ (at 37° C., 20 MHz); Gd-[HP-DO3A]: 3.65 mM⁻¹s⁻¹ (at 40° C., 20 MHz); Gd[DO3A-butrol]: 5.6 mM⁻¹s⁻¹ (at 37° C., 20 MHz)) (see, Caravan, et al., Chem. Rev. 1999, 99, 2293). The enhanced thermodynamic stability and kinetic inertness of these macrocyclic complexes are not obtained at the cost of their relaxivity.

As previously mentioned, the most practical way to improve the sensitivity of MRI and promote its application, especially in molecular imaging, is the lengthening τ_{R} of target MRI CAs. Compared with the recently reported contrast agents based on macromolecular or supramolecular molecules, such as fullerenes (see, for example, Kato et al., J. Am. Soc. Chem, 2003, 125, 4391-4397; Mikawa et al., Bioconjugate Chem., 2001, 12, 510-514; U.S. Pat. App. Pub. No. 2003/0220518 A1 and U.S. Pat. No. 6,471,942 B1), dendrimers (see, for example, Kobayashi et al., Bioconjugate Chem., 2003, 14, 388-394; Sato et al., Magn. Reson. Med., 2001, 46, 1169-1173; and U.S. Pat. App. Pub. No. 2004/0037777 A1), and micellar aggregates (see, for example, Andre, et al., Chem. Eur. J. 1999, 5, 2977-2983; Accardo et al., J. Am. Soc. Chem., 2004, 126, 3097-3107), small molecule MRI contrast agents show their particular advantages such as excellent water solubility, good biocompatibility, low toxicity, and easy excretion from bio-systems. In fact, as previously mentioned, most of the MRI CAs in clinical use are small molecules, for example the well-known [Gd-DOTA(H₂O)] (Dotarem™) and [Gd(DTPA)(H₂O)]²⁻ (Magnevist™). Therefore, a practical and promising way to design the novel MRI contrast agents would be to functionalize the widely used small molecular Gd³⁺ chelates, such as Gd-DTPA or Gd-DOTA, with specific targeting or binding groups. Through this way, not only a higher proton relaxivity of the conjugated adducts can be achieved through the binding with selected macromolecules such as albumin, but also a higher MR signal enhancement at the area of interest as well as significantly lower dosage of contrast agent can also be realized.

Recently, there has been growing interest in macrocyclic heptacoordinate lanthanide chelates, especially tris N-carboxymethyl-1,4,7,10-tetraazacyclodecane (DO3A). Firstly, in DO3A derivatives, with the exception of three pendant chelating moieties, the remaining NH can be freely derivatized to improve the organ/tissue selectivity (see, Corsi, et al., Chem. Eur. J., 2001, 7, 64), enhance the binding ability to specific macromolecules or increase the enzymatic response ability (see, Moats, et al., Angew. Chem., Int. Ed. Engl., 1997, 36, 726; Nivorozhkin, et al., Angew, Chem., Int. Ed. Engl., 2001, 40, 2903). Secondly, a maximum of two coordinated positions are left for the binding of water molecules in these heptadentate Ln³⁺ complexes, which makes the relaxivity of Gd³⁺ complexes increase effectively because the inner-sphere proton relaxivity is linearly proportional to the number of directly coordinated water molecules (Equation 3). More importantly, the thermodynamic stability of Gd-DO3A [log K=22.0, 298 K] has only a moderately decrease compared to that of Gd-DOTA [log K=24.6, 298 K] (see, Bianchi, et al., J. Chem. Soc., Dalton Trans., 2000, 697). Finally, three chelating moieties with negative charges, such as carboxylate, can neutralize the Ln³⁺. The resulting neutral complexes with low osmolality can effectively reduce the pain and tissue sloughing during the injection process.

### SUMMARY OF THE INVENTION

This invention provides the synthetic procedures for three gadolinium (III) complexes GdL1-GdL3 based on DO3A (1,4,7-tris-acetic acid-1,4,7,10-tetraazacyclododecane), which was functionalized with crown compounds such as aza-15-crown-5, 15-crown-5, quinin alkylated diaza-18-crown-6, respectively in high yields and good regioselectivity. Among the three Gd³⁺ complexes prepared, heptadentate GdL1 that was modified with aza-15-crown-5 displayed the most promising properties as a potential MRI contrast agent. The ¹H NMRD profiles of GdL1 show that it has two inner-sphere coordinated water molecules, and the proton relaxivity, r₁, of this complex is significantly higher (9.65 mM⁻¹s⁼¹) than that of the clinically used [Gd-DOTA(H₂O)]⁻ (4.74 mM⁻¹s⁻¹) and the heptadentate complex [Gd-DO3A(H₂O)₂] (5.72 mM⁻¹s⁻¹) with similar two bound water molecules at 20 MHz close to the common magnetic field of clinical MRI under physiological pH ranges. The variable-temperature behavior of ¹⁷O NMR of GdL1 gives a favorable exchange lifetime, τ_{M}, of the inner-sphere coordinated water molecules as 55 ns at 298 K. This result is very close to its optimal value of 20-30 ns at 298 K in theory, and much shorter than that of [Gd-DOTA(H₂O)]⁻ 245 ns and Gd-DO3A(H₂O)₂] 160 ns Significantly, GdL1 shows very promising targeting-specificity to the kidney and liver. Renal (up to 99%) and hepatic (up to 57%) intensity enhancements were confirmed after the administration of this complex, even at a dose of 20 μmol/kg, which is only ⅕ of the typical clinical dosage (100 μmol/kg) of [Gd-DOTA(H₂O)]⁻. Moreover, the strong renal, and especially the hepatic intensity enhancements induced by GdL1 are maintained for more than four hours, much longer than that of other small molecule MRI CAs such as Gd-DOTA. GdL2 and GdL3 modified with different crown ethers also showed a similar targeting-specificity to the kidney and liver, and prolonged resident lifetimes in these organs. Therefore, the common shortcoming of small molecule MRI contrast agents, rapid excretion rate, can be overcome. Furthermore, the cytotoxicity of GdL1 was evaluated by cell proliferation assay in vitro, and the viability of the MIHA cells was not affected obviously even in the presence of a high concentration of this compound (1×10⁻² M) under a long incubation time (24 h). In conclusion, the efficient S renal and hepatic intensity enhancement, high tissue/organ specificities, long resident lifetime, low dosage requirement and low cytotoxivity make these complexes with pendant crown compounds, especially GdL1, very promising potential MRI contrast agents.

### BRIEF DESCRIPTION OF THE DRAWINGS

FIG. 1 is a diagram showing schematic structures of the interaction between the Gd³⁺ ion and the proximate water protons, and the relationship between various parameters that determine the relaxivity of Gd³⁺ complex. The relaxivity of a Gd³⁺ chelate is governed by the dipolar interaction between the metal ion and the proximate water protons. τ_{M}^{O-1} is the exchange rate of the inner-sphere water, τ_{M}^{H-1} is the exchange rate of its protons, τ_{R} is the rotation correlation time, kₑₓ is the water/proton exchange rate, τ_{M} is the water residence lifetime, T_{iE} (i=1, 2) is the electronic relaxation times of the metal ion, r_{H} and a are the distance between the metal ion and the bound water protons, and the bulk water molecules, respectively.

FIG. 2 shows the chemical structures of the clinically approved Gadolinium (III) MRI contrast agents and Gd-DO3A.

FIG. 3 shows the chemical structures of GdL1-GdL3.

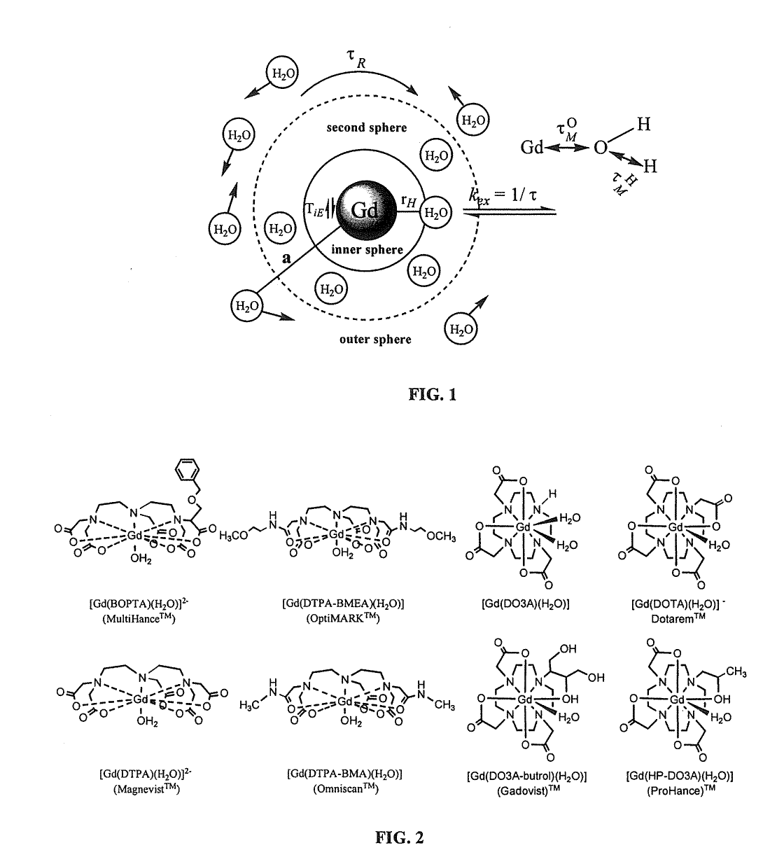

Scheme 1 Two straightforward synthetic procedures to prepare DO3A derivatives by selective N-alkylation.

Scheme 2 Synthetic procedure for the preparation of GdL1. Preparation of GdL1. ^{a}Reagents and conditions: (a) chloroacetyl chloride, (Et)₃N/CH₂Cl₂, r.t., 2 h, 88%; (b) 2.5 equiv. cyclen, K₂CO₃/CH₃CN, 50-60° C., 12 h, 86%; (c) BH₃.THF/THF, reflux, 12 h, 81%; (d) 3.3 equiv. tert-butyl bromoacetate, Na₂CO₃/THF—H₂O, r.t., 4 h, 84%; (e) TFA/CH₂Cl₂, r.t., 2 h, 96%; (f) Gd₂(CO₃)₃, H₂O, 80° C., 10 h, 92%.

Scheme 3 Synthetic procedure for the preparation of GdL2. ^{a}Reagents and conditions: (a) 2-bromothanol, triethylbenzyl ammonium chloride, KOH/H₂O, 80° C., 12 h, 84%; (b) 3.5 equiv. ted-butyl bromoacetate, (Et)₃N/CHCl₃, r.t., 12 h, 77%; (c) K₂CO₃/CH₃CN, 50-60° C., 12 h, 91%; (d) TFA/CH₂Cl₂, r.t., 2 h, 95%; (e) Gd₂(CO₃)₃, H₂O, 80° C., 10 h, 93%,

Scheme 4 Synthetic procedure for the preparation of GdL3. ^{a}Reagents and conditions: (a) 2-methylchloride quinoline, K₂CO₃/CH₃CN, 60-65° C., 78%; (b) chloroacetyl chloride, (Et)₃N/CH₂Cl₂, r.t., 2 h, 86%; (c) cyclen, K₂CO₃/CH₃CN, 60-65° C., 85%; (d) 3.5 equiv. tert-butyl bromoacetate, Na₂CO₃/THF—H₂O, r.t., 4 h, 90%; (e) TFA/CH₂Cl₂, r.t., 5 h, 92%; (f) Gd₂(CO₃)₃, H₂O, 80° C., 16 hr 92%.

FIG. 4 is a plot of ¹H NMRD profiles of GdL1 in aqueous solution at 25° C. with pH values of 6.9 and 10.0, respectively.

FIG. 5 is a plot of temperature dependence of the water ¹⁷O NMR transverse relaxation rate for GdL1 at 2.12 T and pH 6.0.

FIG. 6 is a plot of ¹H NMRD profile of GdL2 in aqueous solution at 25° C. with pH 6.9 and 10.0 respectively.

FIG. 7 is a plot of temperature dependence of the water ¹⁷O NMR transverse relaxation rate for GdL2 at 2.12 T and pH 6.9.

FIG. 8 is a plot of ¹H NMRD profile of GdL3 in aqueous solution at 25° C. with pH 7.4.

FIG. 9 is a plot of temperature dependence of the water ¹⁷O NMR transverse relaxation rate for GdL3 at 2.12 T and pH 7.4.

FIG. 10 are MR images of a longitudinal section of a rat showing the kidney cortex before (control) and after the administration of GdL1 (0.1 mmol/kg).

FIG. 11 are MR images of a longitudinal section of a rat showing the kidney and liver before (control) and after the administration of GdL1 (0.1 mmol/kg).

FIG. 12 are MR images of a transverse section of a rat showing the kidney (cortex and medulla) before (control) and after the administration of GdL1 (0.1 mmol/kg),

FIG. 13 are MR images of a traverse section of a rat showing the liver before (control) and after the administration of GdL1 (0.1 mmol/kg).

FIG. 14 is a plot of time dependence of the GdL1 (0.1 mmol/kg) induced hepatic and renal intensity enhancements.

FIG. 15 is a plot of time dependence of the GdL1, Gd-DOTA, and Gd-DO3A (standard dose, 0.1 mmol/kg) induced renal (cortex) intensity enhancements as obtained from the T₁-weight images.

FIG. 16 is a plot of time dependence of the GdL1, Gd-DOTA, and Gd-DO3A (standard dose, 0.1 mmol/kg) induced hepatic intensity enhancements obtained from the T₁-weight images.

FIG. 17 are MR images of a longitudinal section of a rat showing the kidney before (control) and after the administration of GdL1 at ⅓ of the standard dosage (0.033 mmol/kg).

FIG. 18 are MR images of a longitudinal section of a rat showing the liver before (control) and after the administration of GdL1 at ⅓ of the standard dosage (0.033 mmol/kg).

FIG. 19 are MR images of a transverse section of a rat showing the kidney and liver before (control) and after the administration of GdL1 at ⅕ of the standard dosage (0.02 mmol/kg).

FIG. 20 is a plot of time dependent renal intensity enhancements induced by ⅓ and ⅕ of the standard dosage of GdL1 (0.033 mmol/kg and 0.02 mmol/kg) compared with that of Gd-DOTA at a standard dosage.

FIG. 21 is a plot of time dependent hepatic intensity enhancements induced by ⅓ and ⅕ of the standard dosage of GdL1 (0.033 mmol/kg and 0.02 mmol/kg) compared with that of Gd-DOTA at a standard dosage.

FIG. 22 shows MIHA cells that were stained by AO/EB, and observed under laser scanning confocal microscopy. MIHA cells in the absence (control) and presence of Gd-DOTA (10 μM), Gd-DO3A (10 μM), and GdL1 (10 μM) incubated at 37° C. and 5% CO₂, 95% air in a humidified incubator for 1 h, 2 h, 8 h, and 24 h, respectively.

FIG. 23 are plots of % viability vs. dosage concentrations of GdL1, Gd-DOTA, and Gd-DO3A after 1 h, 2 h, 8 h, and 24 h incubation.

FIG. 24 is a plot of time dependence of the GdL2 (0.1 mmol/kg) induced renal and hepatic intensity enhancements.

FIG. 25 is a plot of time dependence of the GdL3 (0.1 mmol/kg) induced renal and hepatic intensity enhancements.

### DETAILED DESCRIPTION OF THE INVENTION

In order to facilitate the review of the various embodiments of the invention, the following explanations of specific abbreviations and terms are provided:

1. Abbreviations

MR—magnetic resonance

MRI—magnetic resonance imaging

DTPA—diethylenetriaminepentaacetic acid

DOTA—1,4,7,10-tetra-(acetic acid)-1,4,7,10-tetraazacyclododecane

DO3A—1,4,7-tris-(acetic acid)-1,4,7,10-tetraazacyclododecane

VT—variable temperature

CAs—contrast agents

NMRD—nuclear magnetic resonance dispersion

SB—Solomon-Bloembergen

BM—Bloembergen-Morgan

ZFS—zero-field splitting

DTPA-BMA—diethylenetriamine-N,N,N′,N″,N′″-pentaacetate-bistmethylamide)

BOPTA—4-carboxy-5,8,11-tris(carboxymethyl)-1-phenyl-2-oxa-5,8,11-triazatridecan13-oic acid

EOB-DTPA—(S)-N-[2-[bis(carboxymethyl)-amino]-3-(4-ethoxyphenyl)propyl]-N-[2-[bis(carboxymethyl)amino]-ethyl]glycine

2. Terms

Unless otherwise explained, all technical and scientific terms used herein have the same meaning as commonly understood by one of ordinary skill in the art to which this invention belongs. The singular terms “a”, “an”, and “the” include plural referents unless the context clearly indicates otherwise. Similarly, the word “or” is intended to include “and” unless the context clearly indicates otherwise. The term “comprises” means “includes”.

This invention concerns novel MRI contrast agents based on metal chelators functionalized with crown compounds, which show enhanced water proton relaxivities and targeting-specificity to the kidney and liver in vivo with the advantages of excellent water solubility, high tissue/organ specificities, efficient intensity enhancement, long resident lifetime, a low dosage requirement, and low toxicity.

Crown compounds are generally described as macrocyclic compounds that have hetero atoms such as O, N, or S as the electron donor atoms in their ring structures, and the property of incorporating cations into their cavities. Macrocyclic polyethers that have only O atoms as the donor atoms are termed crown ethers. Cyclic amino ethers in which N (NH, NR) substitutes for some of the O donor atoms of crown ethers are known as azacrown ethers. Moreover, the multicyclic crown compounds, whose two bridgeheads consist of two N groups, are termed as cryptands. The common names of these ethers include a number as a prefix to designate the total number of atoms in the ring, and a number as a suffix to designate the number of oxygen atoms in the ring. Crown ethers have aroused great interest in recent years because of their important characteristics shown in chemistry and in biology. For example, crown ethers involve the complexation of the ether oxygens with various ionic species, which is termed “host-guest” chemistry, with the ether as host and the ionic species as guest. Furthermore, the structures of some macrocyclic polyethers are similar to those of certain naturally-occurring macrocyclic antibiotics, such as valinomycin, which affect cation transport across biological and artificial membranes. Thus, crown ethers mimic, in a relatively uncomplicated way, the very complicated functions of biological materials such as enzymes (Naumowicz at al., Cell. Mol. Biol. Let., 2003, 8, 383-389). The study of crown compounds may indicate new approaches in the development of pharmaceutical systems, or a way to cross the blood-brain barrier, which may help to explain how the body moves some critical bio-cations or small molecules.

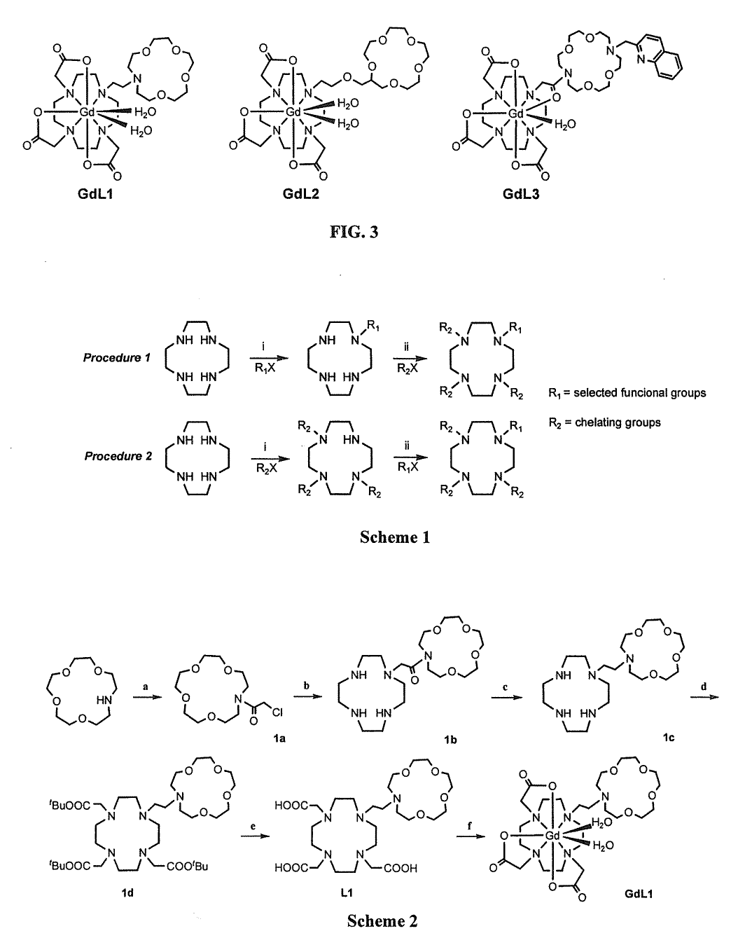

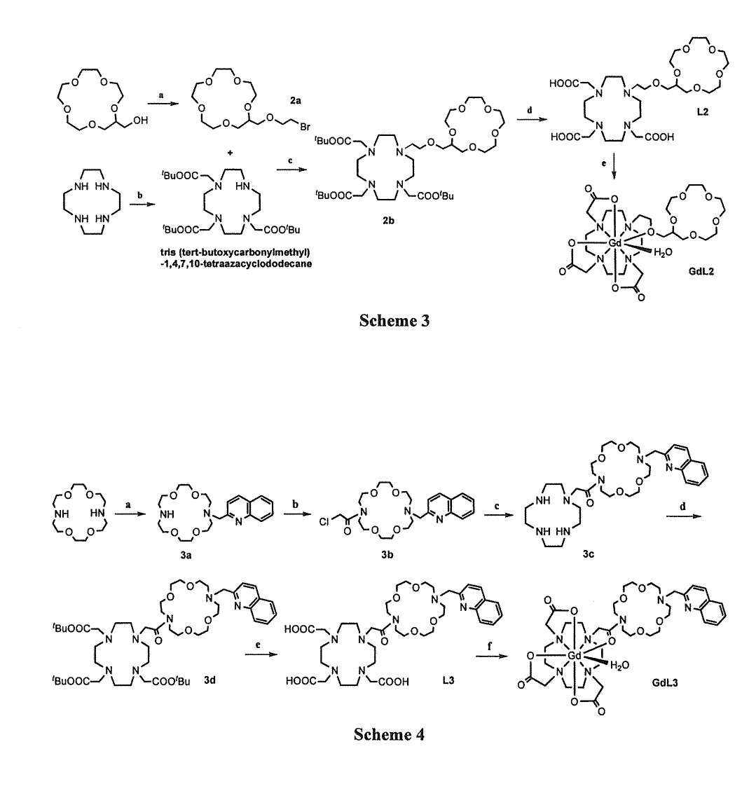

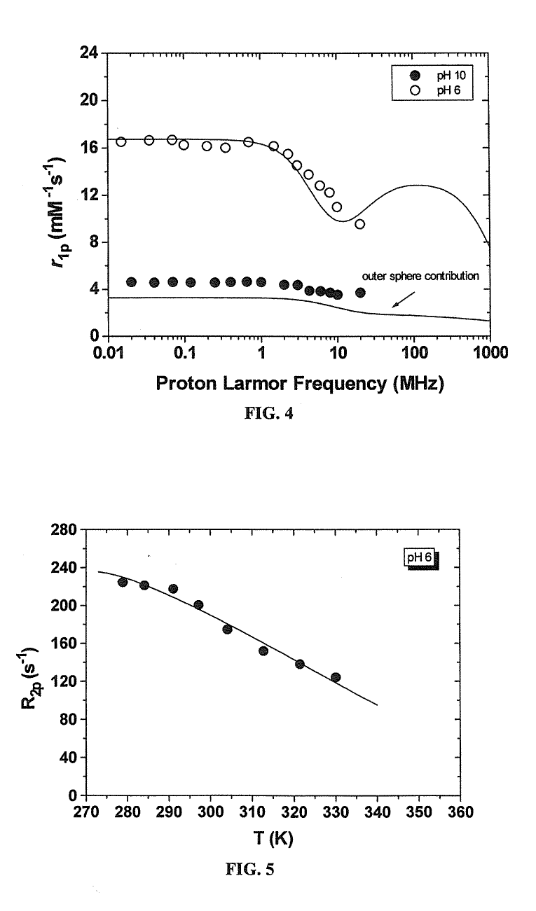

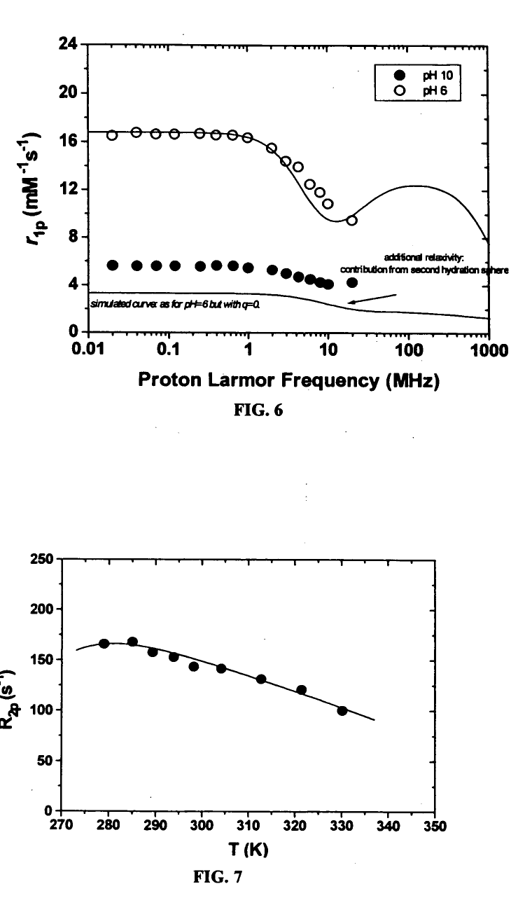

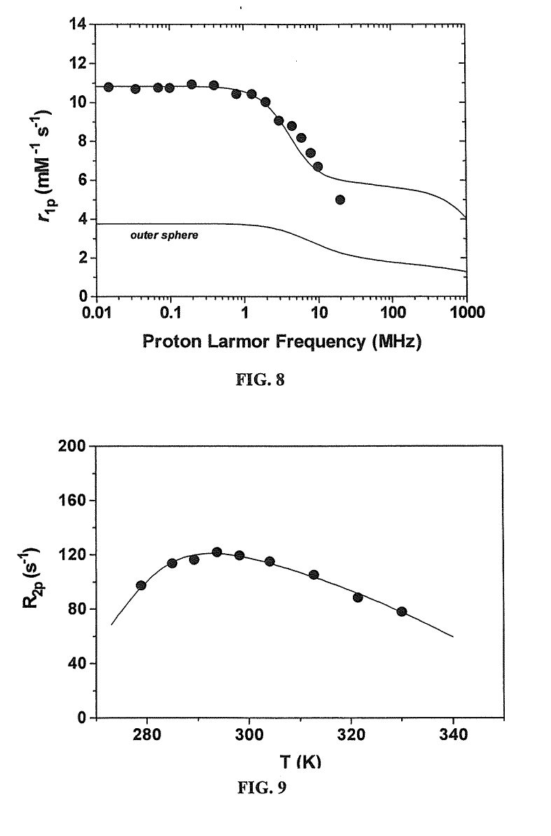

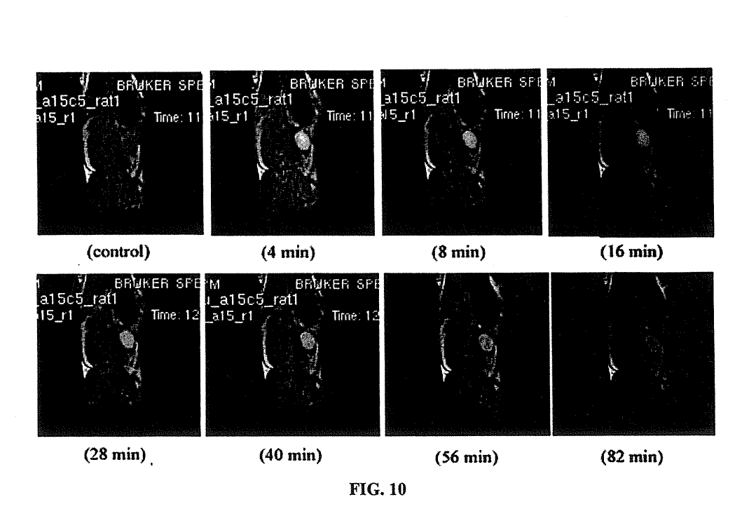

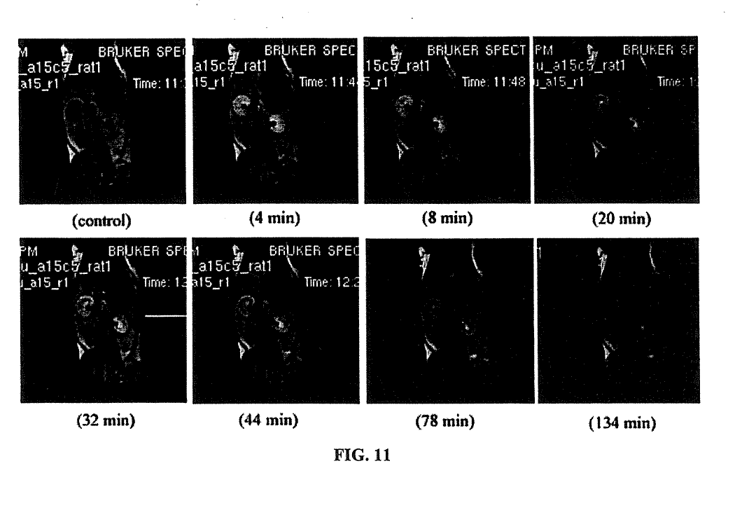

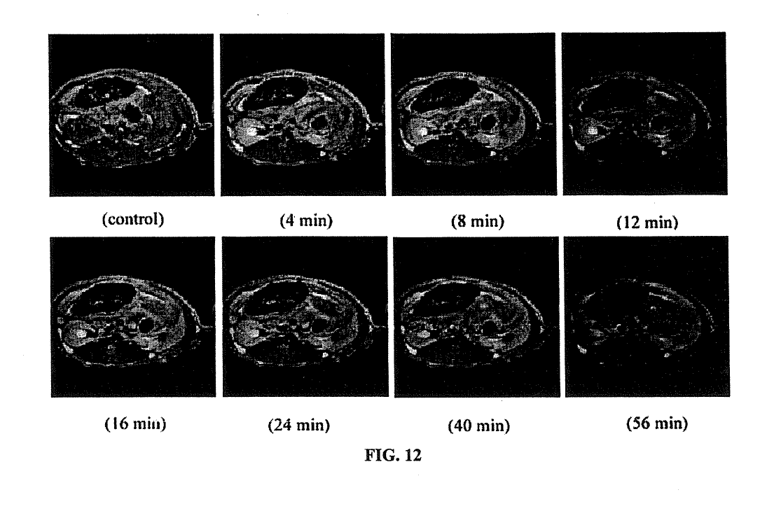

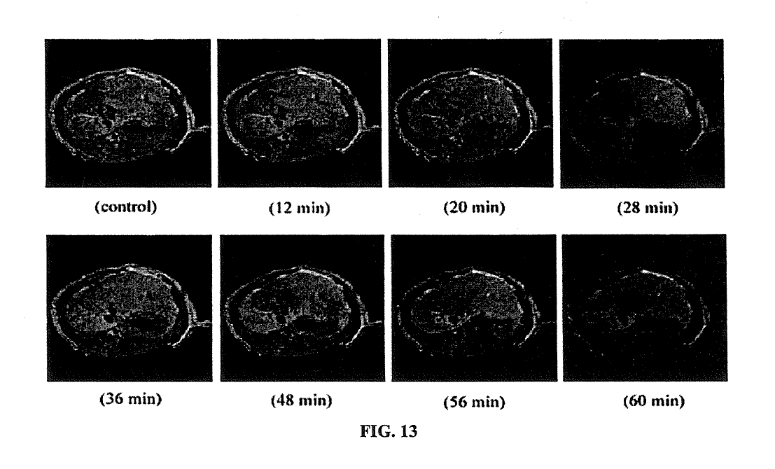

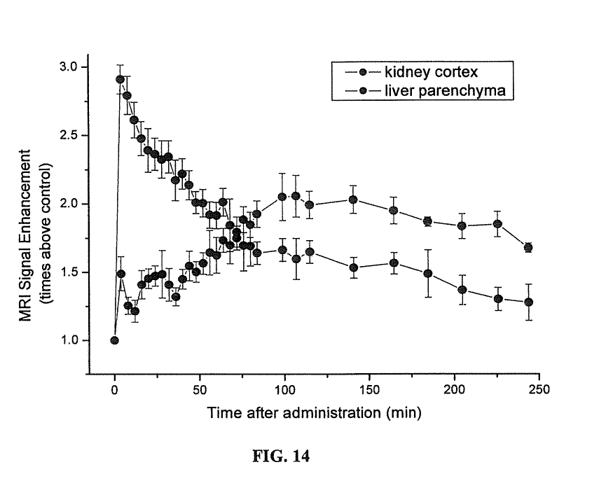

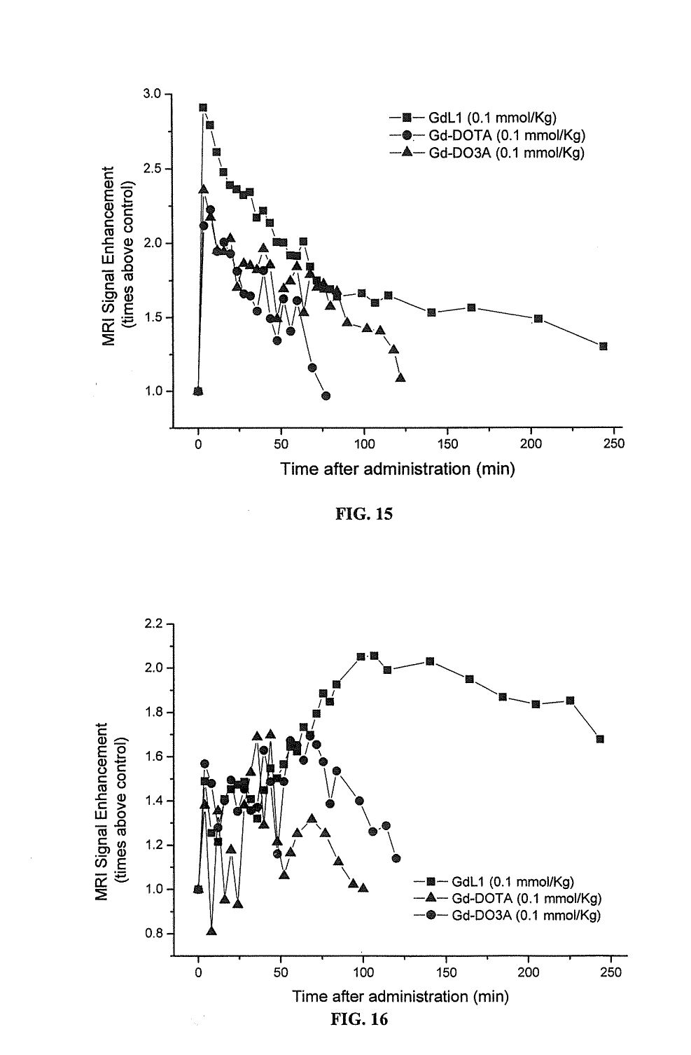

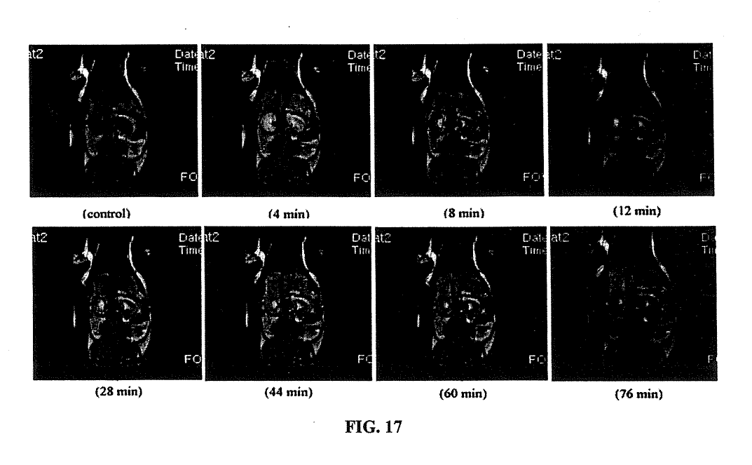

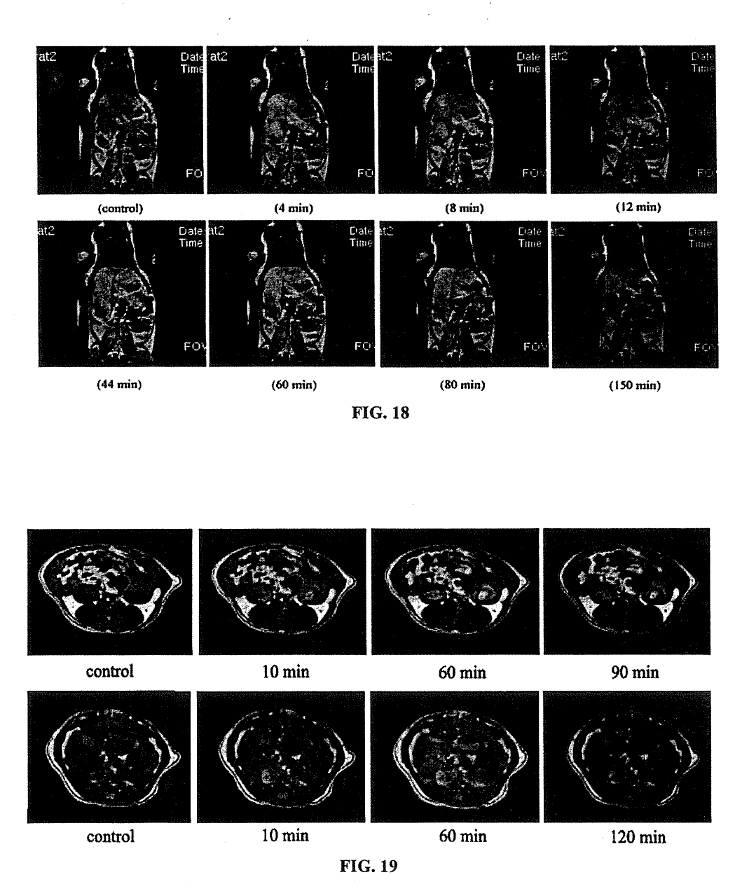

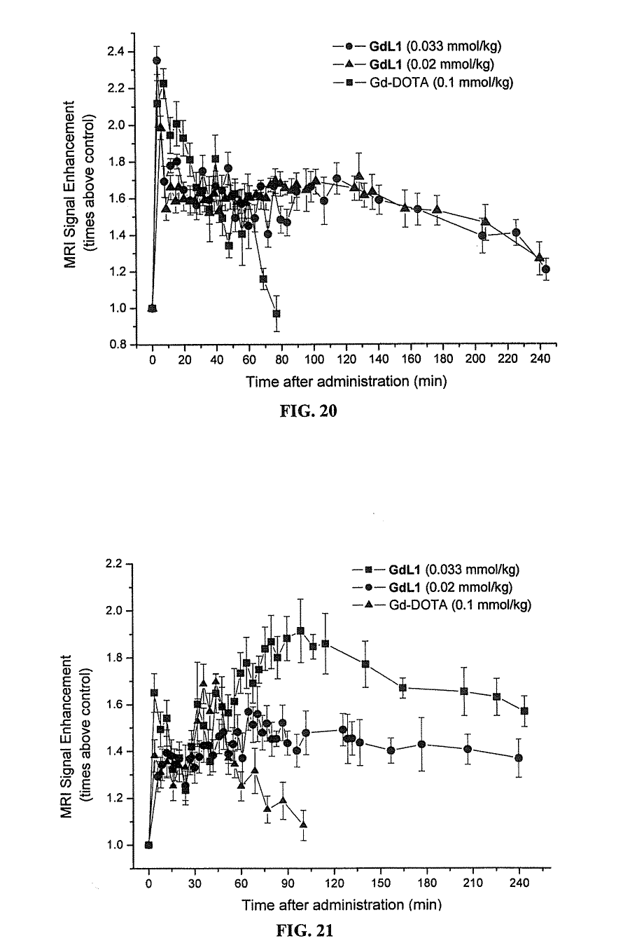

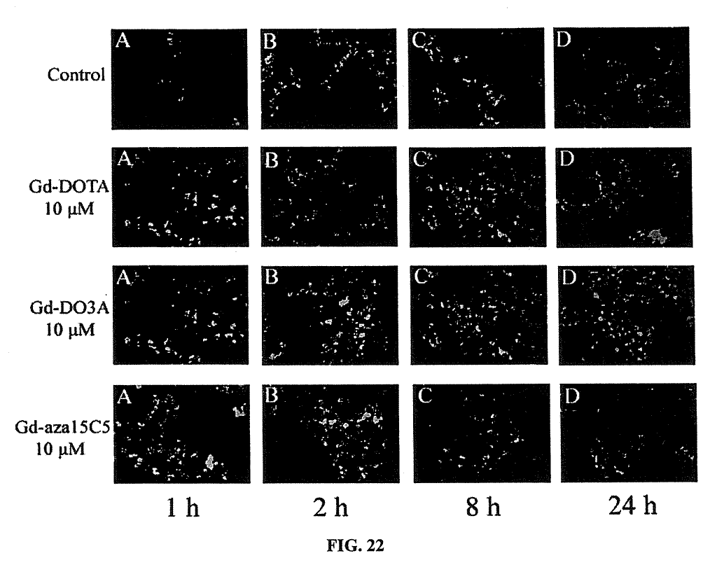

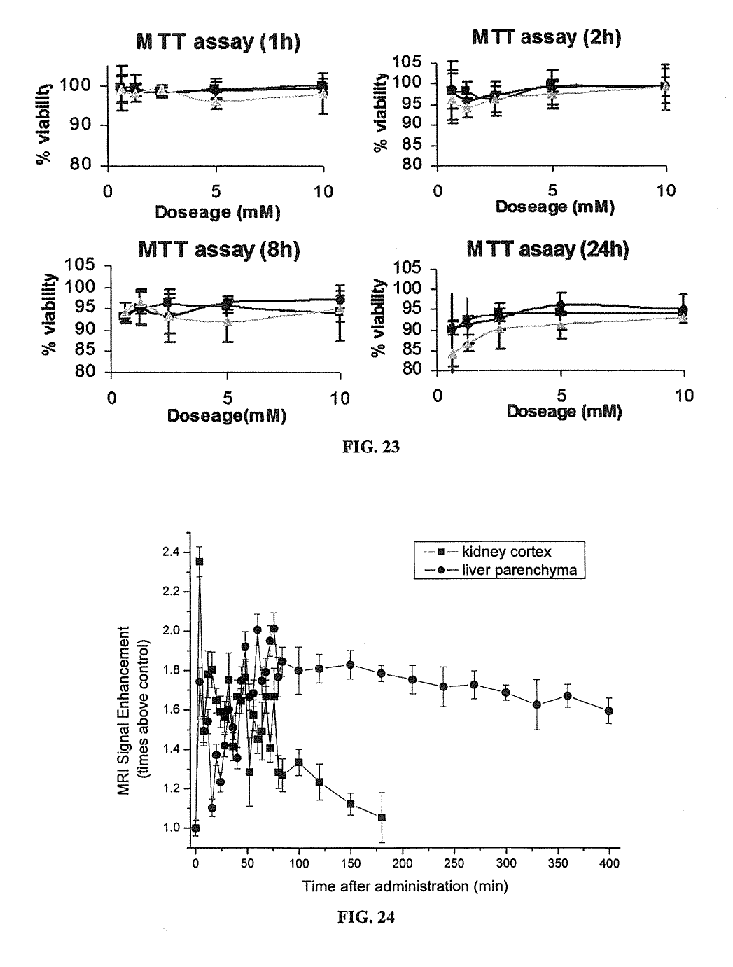

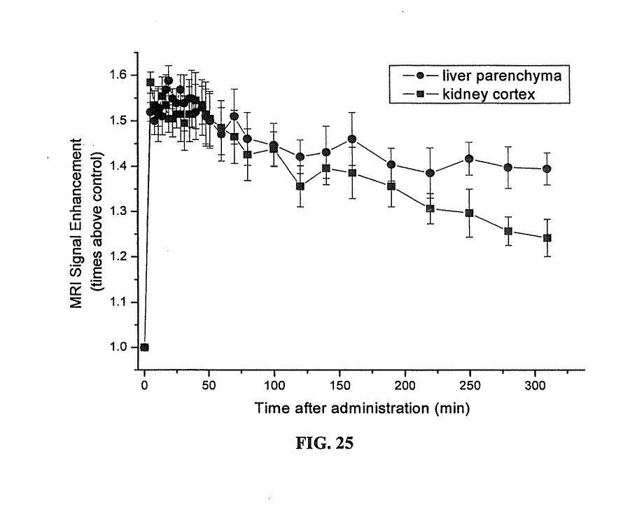

Most of the investigations that are related to crown ethers focus on the roles that they play in host-guest chemistry or supramolecular chemistry. Very rarely are examples given for the biological activity of crown ethers in physiological background (Leong, B. K. J., Chem. Eng. News, 1975, 5). In this invention, we found that pendant crown compounds can significantly enhance the targeting-specificity to the kidney and especially the liver in vivo, and can prolong the resident lifetime of the paramagnetic complexes in these two organs. Three different crown ethers, including aza-15-crown-5 (L1), 15-crown-5 (L2), and quinine alkylated diaza-18-crown-6 (L3) were introduced to the DO3A chelate (FIG. 3).

Ligand L1 was prepared using the synthetic procedures and reaction conditions described in Scheme 2 with satisfactory yields. Following the straightforward alkylated procedure 1 (Scheme 1), treatments of the electrophiles N-chloroacetyl-aza-15-crown-5 (1a) with 2.5 equiv. cyclen in CH₃CN/K₂CO₃ gave mono N-alkylated products 1b with high regioselectivity (Li et al., Tetrahedron Lett. 2002, 43, 3217-3220). The amide in 1b was reduced to amine in the presence of BH₃.THF in succession. The resulting products 1c reacted with excess tert-butyl bromoacetate in THF/Na₂CO₃ to afford the corresponding alkylated products 1d. Through the deprotection step in TFA/CH₂Cl₂, the ligand L1 was obtained in satisfactory yield. The treatment of L1 with Gd₂(CO₃)₃ in aqueous solution gave the targeting complex GdL1.

L2 was prepared according to “procedure 2” in Scheme 1 due to the low yields of electrophile 2a. The treatment of hydromethyl-15-crown-5 with an excess of 2-bromothanol in the aqueous solution of KOH in the presence of triethylbenzyl ammonium chloride as a phase-transfer catalyst afforded the corresponding electrophile (Scheme 3). Then, the halide 2a was reacted with tris (tert-butoxycarbonylmethyl)-1,4,7,10-tetraazacyclododecane (detailed synthetic procedure for this compound, see U.S. Pat. provisional application No. 60/485,219, filed on Jul. 8, 2003; Li et al., Tetrahedron, 2004, 60, 5595-5601) in CH₃CN/K₂CO₃ to give the ligand precursor 2b. After deprotection in TFA/CH₂Cl₂, the ligand L2 was obtained in a satisfactory yield. Finally, the complex GdL2 was achieved by complexing the ligand and Gd₂(CO₃)₃.

Ligand L3 was prepared according to Scheme 4. 2-methylchloride quinoline was reacted with 1.2 equiv. of 1,4,10,13-tetraoxa-7,16-diaza-cyclooctadecane to give the mono N-alkylated product 3a, which was further treated with chloroacetyl chloride to obtain 3b in good yield. Mono N-alkylated cyclen 3c was achieved by the reaction between electrophile 3b and 3.0 equiv. cyclen. The chelating groups were then introduced to other three NH positions. After deprotection, the resulting ligand L3 was treated with Gd₂(CO₃)₃ to afford the ultimate GdL3. GdL3 was synthesized with two aims. The first was to investigate the effect of ring size of pendant crown ether to the targeting-specificity of this Gd complex in vivo. The second was to improve the targeting-specificity and sustained MRI signal enhancement in cardiac-vascular tissue and liver parenchyma using hydrophobic functional group such as aromatic groups (for example, [Gd(BOPTA)(H₂O)]²⁻ (MultiHance™), and Saab-Ismail et al., J. Med. Chem., 1999, 42, 2852-2861). In this case, the organ/tissue specificity and MRI signal intensity enhancement induced by the corporate effect of aza-18-crown-6 and alkylated quinine are worth being measured.

As previously mentioned, inner-sphere proton relaxivity is linearly proportional to the number of water molecules q that are directly coordinated to the Gd³⁺ center. However, there is a trade-off between the q value and toxicity. Due to this concern, it is not feasible to increase the relaxivity significantly by simply increasing the q value. It is noteworthy that there is no direct method of measuring the hydration number. The two commonly used indirect methods are as follows: (1) Lanthanide-induced shift (LIS) methods (see, Alpoim et al., J. Chem. Soc., Dalton Trans., 1992, 463). For paramagnetic Ln³⁺ ions, the plot of the observed ¹⁷O shift in Ln³⁺ chelate solution versus the complex concentration gives a straight line, with the slope being proportional to its hydration number. (2) The lanthanide luminescence method. According to the Horrocks equation, the q value can be calculated by the emission rate constants for the depopulation of Tb³⁺ or Eu³⁺ complexes measured in H₂O and D₂O (see, Beeby at al., J. Chem. Soc., Perkin Trans. 2, 1999, 493). With this method, the q values of the Gd³⁺ complexes in aqueous solution can be measured indirectly by their corresponding Tb³⁺ or Eu³⁺ complexes, because of the very similar coordination behavior between these three lanthanide ions.

TABLE 1
Absolute emission depopulation constants rate (k/ms⁻¹, ±10%)^{b} in
H₂O and D₂O, and hydration states of the complexes TbL1-TbL3,
Tb-DO3A, and Tb-DOTA at pH 6.9 (0.1 M Tris/HCl), 298 K.

| | | | | | Tb- |
|---|---|---|---|---|---|
| Complex | TbL1 | TbL2 | TbL3 | Tb-DO3A | DOTA |
| k_{H2O} | 0.72 | 0.74 | 0.50 | 0.88 | 0.48 |
| k_{H2O} | 0.34 | 0.35 | 0.31 | 0.42 | 0.27 |
| q^{a} | 1.6 | 1.7 | 0.7 | 2.0 | 0.8 |

^{a}Values of q were derived using: q^{Tb} = 5 (1/τ_{H2O} − 1/τ_{D2O} − 0.06) after correcting for the estimated effect of unbound water molecules. ^{b}k = 1/τ_{H2O}.

The luminescent measurements confirm that TbL1 and TbL2 have a similar heptadentate coordination structure, with two inner-sphere coordinated water molecules at pH 6.9. However, there is only one bound water molecule that can be detected for the complex TbL3 with the octadentate coordination mode. In TbL3, the carboxyl in the amide replaces one of the bound water molecule, and coordinates with the Tb³⁺ centre (FIG. 3).

Measuring the relaxation rates of an abundant nuclear species as a function of the magnetic field over a wide range is called relaxometry. A relaxometry profile is a plot of the nuclear magnetic relaxation rate, usually 1/T₁, as a function of the Larmor frequency or the magnetic field. This profile is also called a Nuclear Magnetic Resonance Dispersion (NMRD) profile. Compared with the NMRD profile, the prominent advantage of the ¹⁷O NMR technique is that it allows the accurate determination of the water exchange rate 1/τ_{M}. This is a consequence of the oxygen nucleus being closer to the paramagnetic centre when bound in the inner-sphere, wherein the outer-sphere contributions to the relaxation rates are negligibly small. With the help of NMRD profiles, ¹⁷O NMR measurements, and the Soloman-Bloemnergen-Morgan theory, several important parameters, such as τ_{M}, τ_{R}, τᵥ, Δ² and τ_{H}, can be measured.

The efficacy of a paramagnetic complex in the enhancement of the nuclear magnetic relaxation of solvent protons is routinely expressed in terms of relaxivity r₁ₚ, i.e. the increase in the water proton longitudinal relaxation rate in a 1 mM solution of the contrast agent. Typically, for Gd³⁺ complexes such as Gd-DOTA and Gd-DTPA with one coordinated water molecule, the r₁ₚ is 4.74 and 4.69 mM⁻¹s⁻¹, respectively (at 20 MHz, 298 K), and their bound water molecular resident lifetime τ_{M} are about 244 ns and 303 ns respectively (298 K). However, even though the relaxivity (inner-sphere component) is directly proportional to the number q of metal-bound water molecules, heptadentate Gd³⁺-DO3A derivatives with two water molecules have seldom been measured. In this invention, complex GdL1 with pendant aza-crown ethers was investigated by 1/T₁ NMRD profiles and variable-temperature ¹⁷O NMR measurements respectively. Moreover, Gd-DOTA and Gd-DO3A as the parent molecules were also measured as comparison.

The NMRD profiles of GdL1 (FIG. 4) were recorded at two different pH values. At 20 MHz and 25° C., the Gd complex shows a relaxivity of 9.65 mM⁻¹s⁻¹ at pH 6.9, which is much higher than the values for Gd-DO3A (5.72 mM⁻¹s⁻¹) and Gd-DOTA (4.74 mM⁻¹s⁻¹) under similar conditions. The large difference in the measured relaxivites of GdL1 and Gd-DOTA can be partially explained by the different hydration numbers of the complexes. At 20 MHz, the relaxivity is mainly dependent on the reorientational correlation time (τ_{R}), which in turn is determined by the molecular weight of the complex.

The molecular weight of GdL1 is 746 Da, higher than that of Gd-DO3A (501 Da), which also partially explains why the relaxivity of GdL1 is even higher than Gd-DO3A with the same inner-sphere bound waters. Furthermore, there is a possible formation of a second coordination shell that is made up by the water molecules hydrogen-bonded with the oxygen atoms on the crown ether ring. The second-sphere makes an additional contribution to the enhancement of the proton relaxation rate (M. Botta, Eur. J. Inorg. Chem., 2000, 399-407).

Due to the importance of the exchange lifetime (τ_{M}) of the coordinated water molecule, a detailed investigation of the variable-temperature behaviour of the ¹⁷O transverse relaxation rate was undertaken at 2.12 T in the range of 273-340 K (FIG. 5). The results are plotted to represent the best fitting of the data. The R₂ₚ of GdL1 attains its maximum near 270 K, and a q value of 2 is given after the analysis of the data. The most striking result from the data fittings is the water exchange rate of GdL1. At 25° C., compared with the τ_{M} of Gd-DOTA 245 ns (kₑₓ²⁹⁸=4.1×10⁶ s⁻¹), and Gd-DO3A 160 ns (kₑₓ²⁹⁸=6.3×10⁶ s⁻¹), the mean lifetime of GdL1 is 55 ns (kₑₓ²⁹⁸=1.82×10⁷ s⁻¹), which is very close to the optimal value of about 20-30 ns in theory. Table 2 lists the relaxation parameters obtained from the fits of NMRD profile and ¹⁷O NMR transverse relaxation rate.

Experience shows that τ_{M} has a close relationship with the structures of the complexes. The more stable the eight-coordinate intermediate, the higher the activation energy, and hence the lower the exchange rate. Another important factor that determines τ_{M} is the steric hindrance degree around the bound water molecules. The steric compression induced by the ligand systems forces the water molecules to depart from the binding site quickly. Consequently, the exchange rate increases. Furthermore, a higher negative charge favors the leaving of the water molecule in a dissociative water exchange process, thus shortening the water residence time τ_{M}. In the case of GdL1, we suspect that the relationship between the pendant crown ether and the Gd-DO3A chelate resembles the “cover” and “pot”, in which the aza-crown ether induces the steric hindrance around the axial binding site. Meanwhile, the ether oxygen atoms are partially negatively changed at the neutral pH values. The congested and negatively charged coordination surrounding may commonly cause the fast exchange rate of the bound water molecule.

The ¹H NMRD profile (FIG. 6) of GdL2 is similar to that of GdL1 (FIG. 4), due to their similar molecular weights, structures, and the macrocosmic surroundings around the lanthanide center. At 20 MHz with pH 6.9, the overall water proton longitudinal relaxivity r₁ₚ was found to be 9.36 mM⁻¹s⁻¹, which is slightly smaller than that of GdL1, but is still much larger than the r₁ₚ of Gd-DO3A 5.72 mM⁻¹s⁻¹. The black pots in the NMRD profile correspond to the r₁ₚ of GdL2 at pH 10.0. At 20 MHz, the overall relaxivity of GdL2 decreases to only 4.21 mM⁻¹s⁻¹ at pH 10.0, whereas the outer-sphere relaxivity is 2.1 mM⁻¹s⁻¹, which means that the outer-sphere contribution accounts for about 50% of the overall observed proton relaxivity under highly basic conditions. This is a characteristic of the low molecular weight Gd³⁺ chelate with one bound water molecule at imaging field strengths. It can be visualized that the q value of GdL2 turns from two to one while the pH increases from 6.0 to 10.0.

Similar to the temperature dependence of the ¹⁷O NMR for GdL1 (FIG. 5), GdL2 gives a similar bell-shaped profile (FIG. 7). The q value was about 2 at pH 6.9, and the water exchange rate kₑₓ²⁹⁸ was found to be 8.62×10⁶ s⁻¹ (τ_{M}=116 ns). Even though this number is smaller than that of GdL1 (kₑₓ²⁹⁸=1.82×10⁷ s⁻¹), it is still ideal compared to that of Gd-DOTA (kₑₓ²⁹⁸=4.1×10⁶ s⁻¹) and Gd-DO3A (kₑₓ²⁹⁸=6.3×10⁶ s⁻¹). The relaxation parameters of GdL2 are listed in Table 2. As mentioned before, the water exchange rate relates to the geometry and steric crowding at the water binding site as well as the overall charge of the complex (Corsi, et al., Chem. Eur J. 2001, 7, 1383-1389). Both GdL1 and GdL2 are neutral complexes, so it assumed that the diversities in the steric hindrance induced by the different kinds of crown ethers cause the different τ_{M} values.

The NMRD profile of GdL3 has been recorded at pH 7.5, and is fully consistent with a species with q=1. The relaxivity was measured as 4.95 mM⁻¹s⁻¹ at 20 MHz, pH 7.4. The significant decrease in relaxivity compared with that of GdL1 and GdL2 can be attributed to the single inner-sphere bound water molecule, which is caused by the coordination between the carboxyl of amide and the Gd³⁺ ion center. Temperature dependent ¹⁷O NMR measurement gave the bound water exchange lifetime τ_{M} as 246 ns, which is very similar to that of Gd-DOTA. This result can be explained by the increasing stability of the octadentate coordinated mode. The high activation energy of the structure efficiently decreases the water molecule exchange rate. Furthermore, the reorientation lifetime τ_{R} of GdL3 attained to 210 ps because of its large molecular weight.

TABLE 2
Relaxation parameters obtained for GdL1-GdL3, Gd-DOTA, Gd-DTPA,
and Gd-DO3A at 25° C. and pH 6.9 from fits of the NMRD profile
and ¹⁷O NMR transverse relaxation rate.

| | | T_{V} | T_{M} | T_{R} | | | | D |
|---|---|---|---|---|---|---|---|---|
| Complex | Δ² (s⁻¹) | (ps) | (ns)* | (ps) | r (Å) | q | a (Å) | (cm²s⁻¹) |
| GdL1 | 8.0E19 | 13 | 55 | 146 | 2.98 | 2 | 4.0 | 2.24E−5 |
| GdL2 | 9.0E19 | 13 | 116 | 140 | 2.97 | 2 | 4.0 | 2.24E−5 |
| GdL3 | 3.0E19 | 21 | 246 | 210 | 3.03 | 1 | 4.2 | 2.24E−5 |
| Gd-DOTA | 1.3E19 | 7.7 | 300 | 73 | 3.1 | 1 | 4.0 | 2.24E−5 |
| Gd-DTPA | 4.2E19 | 20 | 250 | 73 | 3.1 | 1 | 4.0 | 2.24E−5 |
| Gd-DO3A | 4.6E19 | 14 | 160 | 66 | 3.15 | 2 | 3.9 | 2.24E−5 |

*Derived from VT ¹⁷O NMR spectroscopy

Based on the information that was obtained from the ¹H NMRD profiles and ¹⁷O NMR transverse relaxation measurements, the relaxivities of complexes GdL1 and GdL2 with two inner-sphere coordinated water molecules are found not only to be higher than that of Gd-DOTA with a single bound water molecule, but also much higher than that of Gd-DO3A with similar two water molecules. This experimental result can be partially attributed to the larger molecular weights of GdL1 and GdL2. Furthermore, there is a possible formation of a second coordination shell that is made up of the water molecules hydrogen-bonded with the oxygen atoms on the crown ether ring. The second sphere makes an additional contribution to the enhancement of the proton relaxation rate.

In viva MRI measurement plays an indispensable role in providing the most intuitive and straightforward way to evaluate the properties of potential contrast agents for practical uses. For example, MRI in preclinical research not only has the ability to capture anatomic images that depict the distribution of potential contrast agents, their resident lifetimes and metabolism in different organs or tissues, but also provides a powerful data collection component that can be used to quantitatively analyze the contrast enhancement efficiency and targeting-specificity of the tested complexes. Furthermore, the image data for the quantitation of potential contrast agents in small animals will provide direct reference for their application in human beings. Due to the outstanding properties such as high relaxivity and excellent water solubilities of GdL1-GdL3 shown in the relaxometic measurements in vitro, the performances of these complexes as potential contrast agents were further assessed in MRI measurements in vivo.

Full body scans were taken at approximately 4 min interval up to 400 min post-injection. The signal intensity of the rat kidney cortex increased shortly after the injection of GdL1 (0.5 mL of 0.1 mmol/kg). FIGS. 10-13 demonstrate the T₁-weighted longitudinal and transverse crossing images of the abdomen of the rat tested, in which the contrasts between the kidney cortex and medulla, and the liver parenchyma before and after the application of GdL1 were observed. The contrast enhancement patterns for the kidney and liver were different. The enhancement of the kidney was very fast, and it attained a maximum in just a few minutes. It decreased rapidly thereafter, but a faint contrast enhancement could still be observed even two hours after the injection. The contrast enhancement of the liver was not distinct at first. The obvious enhancement of the liver was monitored 30 min after administration, and the contrast enhancement of the whole liver parenchyma obtained its maximum within 90-100 min. GdL1 showed quite a long resident lifetime in the liver, and a strong intensity enhancement was maintained for more than 4 hours after administration (FIG. 14). To better evaluate the organ/tissue specificity and intensity enhancement efficiency of GdL1, the time dependent renal and hepatic intensity enhancements were measured after the administration of GdL1, Gd-DOTA, and Gd-DO3A at the same dosages (0.5 mL, 0.1 mmol/kg). For all three Gd³⁺ complexes examined, the maximal values of the renal intensity enhancement appeared just in the first few minutes after the administration. GdL1 gave the highest value, and a nearly 200% renal IE was recorded. In the cases of Gd-DOTA and Gd-DO3A, only 110% and 140% of the maximal IE were observed (FIG. 15). Moreover, the metabolism of Gd-DOTA and Gd-DO3A in the kidney cortex was much faster than that of GdL1. Almost all of the IE was diminished in 70 or 120 minutes after the administration of Gd-DOTA or Gd-DO3A. However, 40% renal IE was still preserved even 250 minutes after the injection of GDL1. It is noteworthy that GdL1 shows excellent specificity for the liver (FIG. 16). 40-60% hepatic IE was obtained for all three Gd³⁺ complexes within a few minutes after administration. In contrast to the relatively rapid metabolic rate of Gd-DOTA and Gd-DO3A in the liver, the maximum value (105%) of GdL1 induced hepatic IE was monitored 90-100 min after the injection. Moreover, the high IE remained at a plateau for more than 4 h. The high hepatic IE and long resident lifetime of GdL1 can be attributed to its targeting-specificity to liver tissue.

Decreasing the dosage of the contrast agents administered without compromising the signal intensity enhancement is the best way to reduce the toxicity brought by the contrast agents, and this requires CAs with high organ/tissue specificities and relaxivity. Due to the outstanding renal and hepatic specificities of GdL1 that were displayed in MRI measurements in vivo, similar experiments were conducted of GdL1 administered in small dosages. FIGS. 17-18 and FIG. 19 give the MR imaging of the kidney cortex and the liver parenchyma before and after the administration of GdL1 at ⅓ (0.033 mmol/kg) and ⅕ (0.02 mmol/kg) of standard dosage. For comparison, MRI measurements were also performed in the presence of Gd-DOTA at a similar dosage (0.1 mmol/kg, 0.033 mmol/kg and 0.2 mmol/kg).

As expected, renal IE induced by GdL1 at small dosages attained its maximum in a few minutes after the administration. Interestingly, the maximal renal IEs (123% and 98%) induced by GdL1 at small dosages (0.033 or 0.02 mmol/kg) were even close to that (123%) of Gd-DOTA at the standard dosage (0.1 mmol/kg), and the resident lifetimes of GdL1 in the kidney cortex were independent of the dosages administered, and much longer than that of Gd-DOTA (FIG. 20). The most striking fact about GdL1 as a potential CA is its excellent specificity for the liver. Even though only one third of the original dose (0.03 mmol/kg) was administered, no significant decrease in hepatic IE was detected. The percentage of IE induced by GdL1 was about 50% just a few minutes after the injection. From 60-100 min after injection, it attained its maximum (90%), and a high hepatic IE (50%) persisted even after 320 min. Furthermore, if the dosage of GdL1 was further decreased to ⅕ of the standard dose (0.02 mmol/kg), then the maximal hepatic IE of 58% appeared 60 min after the administration, and over 40% IE was maintained even after 3 h (FIG. 21). In comparison, the maximum values of hepatic IE induced by Gd-DOTA (70%, 0.1 mmollkg) and (30%, 0.033 mmol/kg) were observed within 30-40 min after administration, but this hepatic IE induced decreased rapidly, and faded away after 100 min. Furthermore, no remarkable hepatic IE was detected if only 0.02 mmol/kg of Gd-DOTA was injected.

It is well-known that the main shortage of small molecule MRI contrast agents such as Gd-DTPA and Gd-DOTA in clinical applications is their aimless diffusion between the blood and extracellular fluid, and their rapid excretion rates through glomerular filtration. This has prompted the development of MRI contrast agents in which lipophilic residues are introduced in the chelate units to develop the hepatospecific agents such as Gd-EOB-DTPA or Gd-BOPTA. Compared to the extracellular CAs, these “lipophilic” complexes are shown to be transported into hepatocytes by passive diffusion through the basolateral membrane (Pascolo et al., Biochem. Biophys. Res. Commun., 1999, 257, 746-752). Recently, MRI CAs with pendant bile acids, which show much higher hepatic specificity and the capability to enter hepatocytes by means of active carrier-mediated transport mechanisms, such as the Na⁺/taurocholate transporter (Anelli, et al., J. Med. Chem., 2004, 47, 3629-3641) attract much attention because this active transporter is not usually expressed in the membrane of certain hepatoma cells (vonDippe et al., J. Blot. Chem., 1990, 265, 5942-5945). For GdL1, even though the precise mechanism that underlies its significant targeting-specificity to the liver has yet to be understood, it seems that the pendant crown ethers play important roles in promoting the uptake of GdL1 into hepatocytes. Firstly, the amphipathic property of crown ethers can increase the passive diffusion efficiency of the Gd³⁺ complexes entering hepatocytes, secondly, through the positive carrier-mediated processes operated by Na⁺/taurocholate cotransporting polypeptide (NTCP) or organic anion transporting proteins (OATP), the Gd³⁺ complexes with pendant crown ethers are assumed to enter the hepatocytes due to the interactions between the bio-active cations such as Na⁺ and crown ethers.

In conclusion, GdL1 affords higher intensity enhancements and prolonged resident lifetime in both the kidney and the liver than Gd-DOTA when the same dosage is administered. Moreover, GdL1 shows excellent specificity to the liver parenchyma, and the hepatic IE induced by a low dosage (0.033 mmol/kg) of GdL1 is even higher than that of Gd-DOTA with three times dosage (0.1 mmol/kg). Furthermore, under this dosage, the high hepatic IE (>60%) of GdL1 stays on a plateau for more than 4 h after administration, which indicates a reasonably long resident lifetime in liver tissue. The efficient hepatic IE, long resident lifetime, and low dosage requirement make the complexes with pendant crown compound very promising potential contrast agents.

To further investigate the potential ability of GdL1 as an NMRI contrast agent, its cytotoxicity was measured by MTT assay. The toxicity of GdL1 was determined from the dose-dependence of surviving cells after exposure to this Gd³⁺ complex at different time intervals. For comparison, the cytotoxicities of Gd-DOTA and Gd-DO3A were measured under the same conditions.

Based on cell morphology and cell membrane integrity, normal, necrotic, and apoptotic cells can be distinguished using laser confocal microscopy. FIG. 22 gives the cell morphology in the absence and presence of Gd-DOTA (10 μM), Gd-DO3A (10 μM), and GdL1 (10 μM) at various time intervals after the incubation. In the absence or presence of the Gd³⁺ complexes, the nuclei of the living cells were stained as bright blue spots in the nucleus. Satisfactorily, no obvious apoptotic or necrotic cells were found in the 24 h of incubation.

FIG. 23 gives the viability of the cells as a function of the dosages of the Gd³⁺ complex at different time intervals in the incubation process. The viability of the cells was not affected by the presence of GdL1, and was maintained above 90% when the concentrations of this complex were increased from 1×10⁻⁵ mol L⁻¹ to 1×10⁻² mol L⁻¹. Moreover, both Gd-DOTA and Gd-DO3A show a similar behavior to that of GdL1 in the plots of viability vs. dosages. Furthermore, the cytotoxicity of GdL1 is also independent of the incubation time. MTT assays were conducted 1 h, 2 h, 8 h, and 24 h after the incubation. The cell viability showed no obvious variation during the whole process, and stayed above 90%. In conclusion, the in vitro MTT assay proved that the cytotoxicity of GdL1 is quite low, even in high concentrations, and is similar to that of Gd-DOTA as the commercially available contrast agent.

In vivo MRI measurements were also performed after the administration of GdL2. FIG. 24 demonstrates the time dependent T₁-weighted renal and hepatic intensity enhancements after the injection of GdL2 at the standard dosage. The renal IE induced by GdL2 was very fast, and attained its maximum (135%) a few minutes after administration. This enhancement faded quickly, and only 30% enhancement was left in less than an hour. Significantly, the hepatic IE induced by GdL2 attained its maximum (100%) in 60 min. after administration, and this high enhancement was maintained (>60%) for more than 4 h. Although GdL2 had a smaller maximal renal IE (135%) and shorter resident lifetime compared with GdL1, the time dependent hepatic IE induced by these two Gd³⁺ complexes were very similar. The common feature of hepatic IE induced by GdL1 and GdL2 further demonstrated the important role played by the pendant crown ethers in strengthening the targeting-specificity to the liver.

FIG. 25 gives the time dependent T₁-weighted renal and hepatic intensity enhancements after the administration of GdL3 at a standard dosage. Quite different than GdL1 and GdL2, both the renal and hepatic IEs reached their maximums (58% and 59% respectively) in a few minutes after the injection of GdL3. The IE induced by GdL3 was much lower than that of GdL1 and GdL2 at the same dosage. The relatively low MR signal enhancements induced by GdL3 can be attributed to its single bound water molecule because the inner-sphere proton relaxivity is linearly proportional to the number of directly coordinated water molecules. It is worth mentioning that, even though a low hepatic IE was recorded, the resident lifetime of GdL3 in liver tissue was quite long, and the hepatic enhancement maintained about 50% for more than 4 h. This fact further proves the function of crown ethers in improving the specificity of the resulting complex to the liver in vivo.

In summary, heptadentate Gd³⁺ complexes with pendant crown ethers, such as GdL1 and GdL2 show outstanding properties as potential contrast agents in MRI measurements in vivo. In the case of GdL1, it not only exhibit a much higher IE for the kidney cortex (200%) and liver parenchyma (105%) than that of Gd-DOTA and Gd-DO3A at the same dosages (0.1 mmol/kg), but also shows a much longer resident lifetime in renal (>3 h) and hepatic tissues (>4 h). Furthermore, in comparison with Gd-DOTA (0.1 mmol/kg, standard dose), a similar maximal renal IE (120%) and a much higher maximal hepatic IE (90%) were achieved, even though a smaller dosage of GdL1 (0.03 mmol/kg, one third of standard dose) was administered. The advantages of GdL1 displayed in MRI measurements such as high intensity enhancements, long residence lifetime and smaller dosage requirements offer it great potential as an MRI contrast agent with targeting specificity to the liver and the kidney.

### THE EXAMPLES

The following examples further illustrate and exemplify the invention, but are in no way intended to limit scope of the invention

General Experiment

All reactions were carried out under an argon atmosphere using dry glassware that had been dried under nitrogen. All of the necessary solvents were purified before use. Chloroform, dichloromethane, DMF, and acetonitrile were distilled from calcium hydride and stored over 4 Å molecular sieves. Pyridine and triethylamine were distilled from calcium hydride and stored over sodium hydroxide. Methanol was distilled from magnesium powder and stored over 4 Å molecular sieves. Powdered K₂CO₃ was predried just before use and stored under Ar. Reactions were monitored by thin-layer chromatography (TLC) using 0.25-mm E. Merck precoated aluminium oxide plates. Visualization was accomplished under UV light or in I₂ atmosphere. Flash chromatography was performed with the indicated solvents using aluminium oxide 90 active neutral (particle size 70-230 mesh). Yields refer to chromatographically and spectroscopically pure compounds unless otherwise stated. ¹H NMR spectra were recorded on a DPX300 Bruker FT-NMR spectrometer and an AV400 Bruker FT-NMR spectrometer with chemical shifts (in ppm) relative to tetramethylsilane; ¹³C NMR was measured at 100 and 75 MHz. Fast Atom Bombardment (FAB) mass spectra and HRFAB-MS were obtained on a Finnigan Mat 95 mass spectrometer, and Electron Spray Ionization (ESI) mass spectra was obtained on a LCQ quadrupole ion trap mass spectrometer with mathanol or water as the carrier solvent. UV-vis spectra were recorded on a Hewlett Packard 8453 UV-vis spectrophotometer.

### Example 1

Synthesis and Characterizations of Compounds 1a-1d, L1, and GdL1

N-Chloroacetyl-aza-15-crown-5 (1a). Aza-15-crown-5 (300 mg, 1.368 mmol) was dissolved in anhydrous DCM (20 mL), triethylamine (207 mg, 2.052 mmol) and then chloroacetyl chloride (231.7 mg, 2.052 mmol) was added. The reaction mixture was stirred at room temperature for 2 h. The organic layer was then washed by water (2×20 mL), dried by Na₂SO₄, and evaporated under reduced pressure. The purification of this material was achieved by column chromatography on aluminium oxide eluting with ethyl acetate:hexane=5:6 (v/v). The product was isolated as colorless oil (279 mg, 1.204 mmol). Yield: 88%. ¹H NMR (400 MHz, CDC₃): δ 4.14 (2H, s), 3.79 (2H, t, J=5.2 Hz), 3.62-3.48 (18H, m); ¹³C NMR (100 MHz, CDCl₃): δ 167.2 (C), 71.6 (CH₂), 70.5 (CH₂), 70.4 (CH₂), 70.3 (CH₂), 70.1 (CH₂), 70.0 (CH₂), 69.4 (CH₂), 68.9 (CH₂), 51.0 (CH₂), 50.1 (CH₂), 41.6 (CH₂); ESI-MS m/z 296.2 (M+H)⁺; HRFAB-MS m/z 296.1257 (M+H)⁺ [Calcd. for C₁₂H₂₃O₅NCl (M+H)⁺, 296.1265].

1-(N-acetyl-aza-15-crown-5)-1,4,7,10-tetraazacyclododecane (1b). 260.0 mg (0.88 mmol) 1a dissolved in 10 mL anhyrous acetonitrile was added dropwise to a mixture of cyclen (378.5 mg, 2.20 mmol, 2.5 equiv.), potassium carbonate (5.0 equivs) in 40 mL warm anhydrous acetonitrile under N₂ atmosphere for approximately half an hour. The mixture was then stirred at 65-70° C. for about 12 h. The solution was filtered under reduced pressure and the filtrate was evaporated to leave a crude oil that was purified by column chromatography on aluminium oxide with DCM:MeOH=100:5 as an eluent. The product 2 was isolated as a colorless oil (326.6 mg, 0.76 mmol, 86%). ¹H NMR (400 MHz, CDCl₃): δ 3.74-3.66 (2H, t, J=6.2 Hz), 3.59-3.47 (14H, m), 3.46-3.36 (6H, m), 2.95-2.93 (1H, br), 2.84-2.81 (1H, br), 2.79-2.70 (7H, m), 2.62-2.54 (7H, m); ¹³C NMR (100 MHz, CDCl₃): δ 171.3 (C), 71.4 (CH₂), 70.5 (CH₂), 70.2 (CH₂), 69.9 (3×CH₂), 69.7 (CH₂), 69.3 (CH₂), 56.9 (CH₂), 56.0 (CH₂), 52.3 (CH₂), 52.1 (CH₂), 49.6 (CH₂), 49.4 (CH₂), 48.5 (CH₂), 48.0 (CH₂), 46.7 (CH₂), 45.6 (CH₂), 45.3 (CH₂); ESI-MS m/z 432.4 (M+H)⁺; HRFAB-MS m/z 432.3193 (M+H)⁺ [Calcd. for C₂₀H₄₂N₅O₅ (M+H)⁺, 432.3186].

1-(2-ethyl-aza-15-crown-5)-1,4,7,10-tetraazacyclododecane (1c). A solution of compound 1b (220.0 mg, 0.51 mmol) in anhydrous THF (15 mL) was added slowly to BH₃.THF (20 mL, 1.0 M, 20 mmol) at room temperature. The reaction mixture was refluxed for 12 h, and the solution was allowed to cool, and excess borane was destroyed by the careful addition of methanol. The THF was removed under reduced pressure and the residue was hydrolyzed in refluxing MeOH—H₂O-conc. HCl (4:1:1) 40 mL for 1 h. The methanol was removed under reduced pressure and the resulting solution was treated with 30% aqueous NaOH until the pH rose to 11-12. The aqueous layer was extracted by DCM (3×50 mL), and the combined organic layers were dried by Na₂SO₄. After the solvent evaporated in vacuo, the resulting residue was purified by column chromatography on aluminium oxide (eluted with DCM:MeOH=100:5). The compound 1c was isolated as a colorless oil (172.7 mg, 0.41 mmol, 81%). ¹H NMR (400 MHz, CDCl₃): δ 5.38-5.03 (3H, br, N—H), 3.65-3.56 (16H, m), 3.01-2.84 (10H, m), 2.63-2.60 (14H, m); ¹³C NMR (100 MHz, CDCl₃): δ 70.1 (2×CH₂), 69.7 (2×CH₂), 69.4 (2×CH₂), 68.9 (2×CH₂), 55.1 (CH₂), 54.3 (2×CH₂), 53.3 (CH₂), 52.2 (2×CH₂), 46.8 (2×CH₂), 46.0 (2×CH₂), 44.3 (2×CH₂); ESI-MS m/z 418.4 (M+H)⁺; HRFAB-MS m/z 418.3387 (M+H)⁺ [Calcd. for C₂₀H₄₄N₅O₄ (M+H)⁺, 418.3393].

1-(2-ethyl-aza15-crown-5)-4,7,10-tris(tert-butoxycarbonylmethyl)-1,4,7,10-tetraazacyclododecane (1d). tert-Butyl bromoacetate (3.3 equiv.) was added to 1-(2-ethyl-aza-15-crown-5)-1,4,8,11-tetraazacyclotetradecane 1c (150.0 mg, 0.36 mmol) in a mixture of THF-water (10:1) and sodium carbonate (4.0 equiv.). After stirring overnight at room temperature, the solvent was evaporated in vacuo. The residue was purified by column chromatography on silica gel (CH₃Cl:CH₃OH=100:15) to give colorless oil (229.8 mg, 0.30 mmol, 84%). ¹H NMR (400 MHz, CDCl₃): δ 3.58-3.35 (16H, m), 3.26-2.17 (6H, s), 3.11-2.64 (24H, m), 1.45 (27H, s); ¹³C NMR (100 MHz, CDCl₃) 170.7 (C), 169.9 (2×C), 82.6 (C), 82.3 (2×C), 69.8 (6×CH₂), 68.9 (2×CH₂), 55.7 (CH₂), 55.6 (CH₂), 55.1 (3×CH₂), 52.6 (CH₂), 51.9 (CH₂), 51.4 (CH₂), 50.9 (CH₂), 50.8 (CH₂), 50.5 (3×CH₂), 50.3 (CH₂), 49.6 (CH₂), 28.0 (3×CH₂), 27.9 (6×CH₂); ESI-MS m/z 760.6 (M+H)⁺; HRFAB-MS m/z 760.5422 (M+H)⁺ [Calcd. for C₃₈H₇₄N₅O₁₀ (M+H)⁺, 760.5436].

1-(2-ethyl-aza-15-crown-5)-4,7,10-tris(acetic acid)-1,4,7,10-tetraazacyclododecane. (1e) A solution of 1d (229.8 mg, 0.30 mmol) dissolved in 15 mL TFA was stirred under the N₂ for about 10 h. The solvent was evaporated, and the residue was redissolved in 3 mL methanol, Diethyl ether (30 ml) was then added dropwise, and the white percipitate was collected by careful centrifugation (171.6 mg, 0.29 mmol, 96%). ¹H NMR (400 MHz, D₂O, pH=7.6): δ 4.12-3.96 (4H, br), 3.74-3.62 (4H, br), 3.60-3.42 (14H, m), 3.41-3.12 (14 H, m), 3.10-2.79 (10H, br). ¹³C NMR (100 MHz, D₂0, pH=7.6): 171.7 (C), 169.9 (C), 169.7 (C), 69.4 (2×CH₂), 68.9 (2×CH₂), 68.4 (2×CH₂), 63.5 (2×CH₂), 54.9 (2×CH₂), 54.8 (3×CH₂), 52.9 (CH₂), 51.9 (CH₂), 51.3 (CH₂), 50.7 (3×CH₂), 48.6 (3×CH₂), 46.4 (CH₂); ESI-MS m/z 592.4 (M+H)⁺; HRFAB-MS m/z 592.3560 (M+H)⁺ [Calcd. for C₂₆H₅₀N₅O₁₀ (M+H)⁺, 592.3558].

Gd³⁺ Complex of L1 (GdL1)

L1 (50.0 mg, 0.085 mmol) was dissolved in 30 mL distilled water. The temperature was controlled at 60° C. and gadolinium carbonate hydrate (49.6 mg, 0.10 mmol) was added. After 5 h, the solution was filtered through a Celite plug to remove the excess terbium carbonate. The water was evaporated in vacuo. The crude product was dissolved in methanol, and diethyl ether was added carefully to obtain the product as a white solid, which was very hygroscopic and was stored in a desiccator under inert gas. Yield: 92% (58.4 mg, 0.078 mmol); ESI-MS m/z 747.3 (M+H)⁺; HRFAB-MS m/z 747.2545 (M+H)⁺ [Calcd. for C₂₆H₄₇N₅O₁₀Gd (M+H)⁺, 747.2563]; Calcd. For C₂₆H₄₆N₅O₁₀Gd.5 H₂O: C 37.35, H 6.75, N 8.38%, found: C 37.48, H 6.96, N 8.04%.

### Example 2

Synthesis and Characterizations of Compounds 2a-2d, L2, and GdL2

2-(2-bromoethoxyl)-methyl-15-crown-5 (2a). 300.0 mg (1.2 mmol) of hydromethyl-15-crown-5 and 75.0 mg (0.4 mmol) triethylbenzyl ammonium chloride were added to 10 mL 2-bromothanol at the same time. Then, 5.0 mL 50% sodium hydroxide aqueous solution was also added to this solution and stirred vigorously at 70° C. for about 12 h. After the mixture was cooled down, 10 mL CH₂Cl₂ and 10 mL H₂O was added respectively. The organic phase was collected, and the solvent was evaporated to give the crude product as a yellow oil, which was purified by column chromatography on aluminum oxide (neutral, 70-230 mesh) with ethyl acetate:hexane=7:3 as an eluent to yield a pale viscous oil 1a (287 mg, 0.8 mmol, 67%). ¹H NMR (400 MHz, CDCl₃): δ 3.64 (23H, m), 3.44 (2H, t, J=6.0 Hz). ¹³C NMR (100 MHz, CDCl₃); δ 78.7 (CH), 71.5 (CH₂), 71.4 (CH₂), 71.2 (CH₂), 71.1 (CH₂), 70.9 (CH₂), 70.8 (CH₂), 70.7 (CH₂), 70.6 (CH₂), 70.5 (CH₂). 70.4 (CH₂), 70.3 (CH₂), 30.5 (CH₂); ESI-MS: m/z 357.2 (M+H)⁺, 379.2 (M+Na)⁺; HRFAB-MS m/z 379.0730 (M+Na)⁺ [Calcd. for C₁₃H₂₅O₆BrNa (M+Na)⁺, 379.0732].

1-(2-ethoxylmethyl-15-crown-5)-4,8,11-tris(tert-butoxycarbonylmethyl)-1,4,8,11-tetraazacyclodo-decane (2b). 2-(2-bromoethoxyl)-methyl-15-crown-5 2a (167.0 mg, 1.2 equiv.) and tris(tert-butoxycarbonylmethyl)-1,4,8,11-tetraazacyclododecane (200.0 mg, 0.39 mmol) were added to a mixture of acetonitrile (20 mL) and predried potassium carbonate (5.0 equiv.). After stirring about 12 h at 70° C., the solvent was evaporated, and the residue was purified by column chromatography on silica gel (CHCl₃:CH₃OH=100:15) to give a colorless oil. Yield: 265.3 mg, 86%. ¹H NMR (400 MHz, CDCl₃): δ 4.21-3.64 (23H, m), 3.60-2.56 (24H, m), 1.45 (27H, s); ¹³C NMR (100 MHz, CDCl₃): 170.2 (C), 169.9 (C), 164.5 (C), 84.9 (C), 82.1 (C), 81.9 (C), 77.7 (CH), 70.7 (CH₂), 70.3 (CH₂), 70.2 (CH₂), 69.8 (CH₂), 69.7 (CH₂), 69.6 (CH₂), 69.4 (CH₂), 68.4 (CH₂), 68.2 (CH₂), 65.7 (CH₂), 54.3 (CH₂), 54.0 (CH₂), 53.8 (CH₂), 53.6 (CH₂), 53.4 (2×CH₂), 53.3 (CH₂), 52.8 (CH₂), 52.4 (CH₂), 52.0 (CH₂), 51.6 (CH₂), 48.9 (CH₂), 48.8 (CH₂), 28.1 (9×CH₃); ESI-MS: m/z 791.5 (M+H)⁺, 813.4 (M+Na)⁺; HRFAB-MS m/z 791.5388 (M+H)⁺ [Calcd. for C₃₉H₇₅N₄O₁₂ (M+H)⁺, 791.5381].

1-(2-Ethoxylmethyl-15-crown-5)-4,8,11-tris(acetic acid)-1,4,8,11-tetraazacyclododecane (L2).

Compound 2b (79.2 mg, 0.1 mmol) was dissolved in the solution of 5.0 mL anhydrous CH₂Cl₂ and 5 mL TFA under the protection of N₂. The mixture was stirred overnight at room temperature. The solvent was evaporated in vacuo, the residue was taken up in 3.0 mL methanol, and 30.0 mL diethyl ether was added dropwise. The white precipitate was isolated by careful centrifugation. Yield: 57.3 mg, 92%. ¹H NMR (400 MHz, D₂O): δ 3.72-3.60 (6H, m), 3.60-3.45 (20H, m), 3.44-3.22 (13H, m), 2.97-2.92 (4H, m), 2.71-2.68 (4H, m); ¹³C NMR (100 MHz, D₂O): 178.9 (C), 178.8 (C), 168.5 (C), 77.2 (CH), 70.9 (CH₂), 70.1 (CH₂), 70.0 (CH₂), 69.7 (CH₂), 69.6 (2×CH₂), 69.5 (CH₂), 69.4 (CH₂), 68.8 (CH₂), 67.8 (CH₂), 65.6 (CH₂), 54.6 (CH₂), 53.9 (CH₂), 52.9 (2×CH₂), 51.8 (2×CH₂), 50.9 (2×CH₂), 48.5 (2×CH₂), 47.9 (2×CH₂); ESI-MS: m/z 623.5 (M+H)⁺, 645.5 (M+Na)⁺: HRFAB-MS m/z 623.3514 (M+H)⁺ [Calcd. for C₂₇H₅₁N₄O₁₂ (M+H)⁺, 623.3503].

Gd³⁺ complex of L2 (GdL2). 1-(2-ethoxylmethyl-15-crown-5)-4,8,11-tris(acetic acid)-1,4,8,11-tetraazacyclododecane (L2) (100.0 mg, 0.16 mmol) was dissolved in 30 mL distilled water. The temperature was controlled at 60° C. and gadolinium carbonate hydrate (98.9 mg, 0.20 mmol) was added. After 5 h, the solution was filtered through a Celite plug to remove the excess Gd₂(CO₃)₃. The solvent was evaporated, and the residue was taken up in 3.0 mL methanol and 30.0 mL diethyl ether added dropwise. The white precipitate was isolated by careful centrifugation (hygroscopic and stored in a desiccator under inert gas). Yield: 92% (114.3 mg, 0.15 mmol); ESI-MS m/z 778.3 (M+H)⁺; HRFAB-MS m/z 778.2525 (M+H)⁺ [Calcd. for C₂₇H₄₈N₄O₁₂Gd (M+H)⁺, 778.2509]; Calcd. For C₂₇H₄₇N₄O₁₂Gd.3 H₂O: C 39.03, H 6.43, N 6.74%, found: C 38.88, H 6.66, N 6.54%.

### Example 3

Synthesis and Characterizations of Compounds 2a-3d, L3, and GdL3

16-(2-methylquinoline)-1,4,10,13-tetraoxa-7,16-diaza-cyclo-octadecane. (3a). 2-methylchloride quinoline (200 mg, 1.126 mmol) dissolved in 5 mL anhydrous acetonitrile was added dropwise to a mixture of 1,4,10,13-tetraoxa-7,16-diaza-cyclo-octadecane (354.5 mg, 1.35 mmol, 1.2 equiv.), potassium carbonate (5 equiv.) in 40 mL warm anhydrous acetonitrile in an N₂ atmosphere for about an hour. The mixture was stirred at 60-65° C. for about 8 h. The solution was filtered under reduced pressure, and the filtrate was evaporated to leave a crude oil, which was purified by column chromatography on aluminium oxide with ethyl acetate:hexane=120:100 as the eluent. The product was isolated as a light yellow solid (354.4 mg, 0.88 mmol, 78%). ¹H NMR (400 MHz, CDCl₃): δ 8.04-8.02 (1H, d, J=8.6 Hz), 7.86-7.84 (1H, d, J=8.4 Hz), 7.65-7.63 (1H, d, J=7.6 Hz), 7.62-7.59 (1H, d, J=8.5 Hz), 7.50-7.46 (1H, t, J=6.9, 8.4 Hz), 7.32-7.29 (1H, t, J=7.0, 8.2 Hz), 4.50-4.30 (1H, br), 3.81(2H, s), 3.53-3.31 (16H, m), 2.76-2.67 (8H, m); ¹³C NMR (100 MHz, CDCl₃): δ 161.2 (C), 147.3 (C), 136.4 (CH), 129.1 (CH), 128.7 (CH), 127.6 (CH), 127.4 (C), 125.8(CH), 121.4(CH), 70.4 (2×CH₂), 70.0 (2×CH₂), 69.3 (2×CH₂), 69.2 (2×CH₂), 61.3 (CH₂), 55.1 (2×CH₂), 49.0 (2×CH₂); ESI-MS: m/z 404.3 (M+H)⁺; HRFAB-MS m/z 404.2523 (M+H)⁺ [Calcd. for C₂₂H₃₄N₃O₄ (M+H)⁺ 404.2549].

16-(2-methylquinoline)-7-(N-carbamoyl-methylchloride)-1,4,10,13-tetraoxa-7,16-diaza-cycloocta-decane (3b). 16-(2-methylquinoline)-1,4,10,13-tetraoxa-7,16-diaza-cyclooctadecane 3a (700.0 mg, 1.73 mmol) was dissolved in an anhydrous dichloromethane (30 mL), and the solution was left to cool in ice bath. Triethylamine (520.1 mg, 5.02 mmol, 3.0 equiv.) and then chloroacetyl chloride (391.0 mg, 3.46 mmol, 2.0 equiv.) was added. The reaction mixture was further stirred at room temperature for 2 h. The organic layer was then washed with diluted KOH aqueous solution (2×20 mL), dried by anhydrous Na₂SO₄, and evaporated under reduced pressure. Purification of this material was achieved by column chromatography on silica gel eluting with dichloromethane:methanol=200:20 (v/v). The product was isolated as a yellow oil (714.0 mg, 1.49 mmol). Yield: 86%. ¹H NMR (400 MHz, CDCl₃): δ 7.98-7.96 (1H, d, J=8.5 Hz), 7.90-7.88 (1H, d, J=8.4 Hz), 7.65-7.63 (1H, d, J=8.2 Hz), 7.58-7.55 (1H, d, J=8.5 Hz), 7.54-7.51 (1H, t, J=6.9, 7.0 Hz), 7.36-7.32 (1H, t, J=7.0, 6.9 Hz), 4.05 (2H, s), 3.85 (2H, s), 3.64-3.61 (2H, t, J=6.4, 5.6 Hz), 3.57-3.39 (18H, m), 2.80-2.71(4H, m). ¹³C NMR (100 MHz, CDCl₃): δ 167.0 (C), 160.8 (C), 147.4 (C), 136.2 (CH), 129.3 (CH), 128.8 (CH), 127.5 (CH), 127.3 (C), 126.0 (CH), 121.1 (CH), 70.9 (CH₂), 70.8 (CH₂), 70.7 (CH₂), 70.4 (CH₂), 70.0 (CH₂), 69.8 (CH₂), 69.7 (CH₂), 69.4 (CH₂), 62.3 (CH₂), 54.4 (CH₂), 54.3 (CH₂), 49.6 (CH₂), 47.9 (CH₂), 41.5 (CH₂); ESI-MS: m/z 480.3, (M+H)⁺; HRFAB-MS m/z 480.2273 (M+H)⁺ [Calcd. for C₂₄H₃₅N₃O₅Cl (M+H)⁺ 480.2265].

1-{[16-(2-methylquinoline)-7-(N-carbamoyl-methyl)]-1,4,10,13-tetraoxa-7,16-diaza-cyclooctade-cane}-1,4,7,10-tetraazacyclotetradecane (3c). (200 mg, 0.42 mmol) 3b dissolved in 5 mL anhydrous acetonitrile was added dropwise to a mixture of 1,4,7,10-tetraazacyclotetradecane (cyclen) (217.3 mg, 1.26 mmol, 3.0 equiv.), potassium carbonate (5 equiv.) in 40 mL warm anhydrous acetonitrile under the N₂ atmosphere for about an hour. The mixture was stirred at 60-65° C. for about 8 h. The solution was filtered under reduced pressure, and the filtrate was evaporated to leave a crude light-yellow oil, which was purified by column chromatography on aluminium oxide with CH₂Cl₂:MeOH=200:8 as eluent. The product 3c was isolated as a colorless oil (219.8 mg, 0.36 mmol, 85%). ¹H NMR (400 MHz, CDCl₃): δ 67.93-7.91 (1H, d, J=8.5 Hz), 7.82-7.80 (1H, d, J=8.4 Hz), 7.60-7.58 (1H, d, J=8.2 Hz), 7.51-7.49 (1H, d, J=8.5 Hz), 7.47-7.44 (1H, t, J=7.0, 8.3 Hz), 7.30-7.27 (1H, t, J=7.0, 8.1 Hz), 5.62-4.84 (3H, br), 3.78 (2H, s), 3.55-3.33 (22H, m), 2.78-2.63 (12H,m), 2.61-2.53 (8H, m). ¹³C NMR (100 MHz, CDCl₃): δ 171.0 (C), 160.9 (C), 147.3 (C), 136.1 (CH), 129.1 (CH), 128.7 (CH), 127.4 (CH), 127.2 (C), 125.9 (CH), 120.9 (CH), 70.7 (CH₂), 70.6 (CH₂), 70.4 (CH₂), 70.3 (CH₂), 69.9 (CH₂), 69.7 (CH₂), 69.6 (CH₂), 69.5 (CH₂), 62.3 (CH₂), 56.0 (CH₂), 54.2 (2×CH₂), 51.9 (2×CH₂), 48.1 (CH₂), 47.4 (2×CH₂), 47.1 (CH₂), 46.1 (2×CH₂), 45.4 (2×CH₂); ESI-MS: m/z 616.4 [M+H]⁺, 308.8 [M+2H]²⁺; HRFAB-MS m/z 616.4186 (M+H)⁺ [Calcd. for C₃₂H₅₄N₇O₅ (M+H)⁺ 616.4186].

1-{[16-(2-methylquinoline)-7-(N-carbamoyl-methyl)]-1,4,10,13-tetraoxa-7,16-diaza-cyclooctade-cane}-4,7,10-tris(tert-butoxycarbonylmethyl)-1,4,7,10-tetraazacyclotetradecane (3d). tert-Butyl bromoacetate (418 mg, 2.14 mmol, 3.3 equiv.) was added to 3c (400.0 mg, 0.65 mmol) in a mixture of THF-water (5:1) and sodium carbonate (3.0 equiv.). After stirring overnight at room temperature, the solvent was evaporated in vacuo. The residue was purified by column chromatography on silica gel with CH₂Cl₂:CH₃OH=200:25 as the eluent, Product 3d was isolated as a light-yellow oil (560.5 mg, 0.58 mmol, 90%). ¹H NMR (400 MHz, CDCl₃): δ 7.97-7.95 (1H, d, J=8.5 Hz), 7.87-7.85 (1H, d, J=8.3 Hz), 7.65-7.63 (1H, d, J=8.1 Hz), 7.53-7.49 (2H, m), 7.35-7.32 (1H, t, J=7.4, 7.3 Hz), 3.84 (2H, s), 3.59-3.10 (24H, m), 3.06-2.50 (12H,m), 2.48-1.94 (12H, m), 1.28 (27H, s). ¹³C NMR (100 MHz, CDCl₃): δ 172.7 (C), 172.6 (2×C), 171.1 (C), 160.6 (C), 147.4 (C), 136.3 (CH), 129.2 (CH), 128.7 (CH), 127.6 (CH), 127.3 (C), 126.0 (CH), 121.0 (CH₂), 81.7 (C), 81.5 (2×C), 70.7 (CH₂), 70.6 (CH₂), 70.5 (CH₂), 70.2 (CH₂), 70.0 (CH₂), 69.8 (CH₂), 69.7 (CH₂), 69.4 (CH₂), 62.2 (CH₂), 55.6 (2×CH₂), 55.0 (CH₂), 54.3 (2×CH₂), 52.6 (4×CH₂), 48.5 (4×CH₂), 47.8 (2×CH₂), 47.3 (CH₂), 28.1 (3×CH₃), 27.9 (3×CH₃), 27.8 (3×CH₃); ESI-MS: m/z 980.3 [M+Na]⁺, 490.7 [M+Na+H]²⁺; HRFAB-MS m/z 980.6067 (M+Na)⁺ [Calcd. for C₅₀H₈₃N₇O₁₁Na (M+Na)⁺ 980.6048].

1-{[16-(2-methylquinoline)-7-(N-carbamoyl-methyl)]-1,4,10,13-tetraoxa-7,16-diaza-cyclooctade-cane}-4,7,10-tris(acetic acid)-1,14,7,10-tetra-azacyclotetradecane (L3).

Compound 3d (400.0 mg, 0.42 mmol) was dissolved in 5.0 mL anhydrous dichloromethane, then 10.0 mL trifluoroacetic acid was added to the solution under the protection of N₂. This mixture was stirred overnight at room temperature. The solvent was evaporated in vacuo, and the residue was taken up in 3 mL methanol and 30 mL diethyl ether added dropwise. The resulting white precipitate was isolated by careful centrifugation. Yield: 305.2 mg, 0.39 mmol, 92%. ¹H NMR (400 MHz, CDCl₃): δ 8.52-8.50 (1H, d, J=8.5 Hz), 7.72-7.70 (1H, d, J=8.8 Hz), 7.69-7.67 (1H, d, J=8.4 Hz), 7.63-7.61 (1H, d, J=8.5 Hz), 7.59-7.55 (1H, t, J=7.4, 8.2 Hz), 7.38-7.34 (1H, t, J=7.6, 7.6 Hz), 4.59 (2H, s), 3.39 (6H, s), 3.32-2.40 (42H,m). ¹³C NMR (100 MHz, CDCl₃): δ 172.8 (C), 167.9 (2×C), 165.6 (C), 146.3 (C), 145.5 (CH), 140.6 (C), 134.6 (CH), 129.9 (CH), 128.6 (CH), 128.5 (C), 122.4 (CH), 122.2 (CH), 69.7 (CH₂), 69.6 (CH₂), 69.5 (CH₂), 69.2 (CH₂), 68.7 (2×CH₂), 68.0 (2×CH₂), 63.4 (CH₂), 55.3 (CH₂), 54.7 (CH₂), 54.6 (CH₂), 53.0 (2×CH₂), 50.9 (4×CH₂), 48.2 (4×CH₂), 47.4 (CH₂), 47.0 (2×CH₂); ESI-MS: m/z 790.4 [M+H]⁺, 812.4 [M+Na]⁺, 395.7 [M+2H]²⁺; HRFAB-MS m/z 790.4340 (M+H)⁺ [Calcd. for C₃₈H₆₀N₇O₁₁ (M+H)⁺ 790.4351].

Gd³⁺ complex of L3 (GdL3). 1-(N-ethyl-aza-15-crown-5)-4,8,11-tris(acetic acid)-1,4,8,11-tetraazacyclododecane (L3) (50.0 mg, 0.085 mmol) was dissolved in 30 mL distilled water. The temperature was controlled at 60° C. and gadolinium carbonate hydrate (49.5 mg, 0.10 mmol) was added. After 5 h, the solution was filtered through a Celite plug to remove the excess Gd₂(CO₃)₃. The water was evaporated in vacuo. The crude product was dissolved in methanol, and the diethyl ether was added carefully to obtain the product as a white solid, which was very hygroscopic and stored in a desiccator under inert gas. Yield: 92% (58.4 mg, 0.078 mmol). ESI-MS m/z 945.3 (M+H)⁺; HRFAB-MS m/z 945.3371 (M+H)⁺ [Calcd. for C₃₈H₅₇N₇O₁₁Gd (M+H)⁺, 945.3356]; Calcd. For C₃₈H₅₆N₇O₁₁Gd.5 H₂O: C 44.13, H 6.43, N 9.48%, found: C 44.48, H 6.56, N 9.14%.

Relaxometric Measurements

¹H NMRD Profiles

Water proton nuclear magnetic resonance dispersion NMRD profile is used to record the longitudinal water proton relaxation rates as a function of magnetic fields. The method is, in principle, sensitive to all of the parameters that influence relaxivity. The ¹H NMRD profiles were measured over a continuum of magnetic field strength from 0.00024 to 0.24 T (corresponding to 0.01-10 MHz proton Larmor frequency, 1 T≈41.7 MHz) on a Spinmaster FFC (Stelar, Mede (PV)), which is a fast field-cycling NMR relaxometer installed at the LIMA Laboratory (Bioindustry Park, Ivrea (TO), Italy). The relaxometer worked under complete control with an absolute uncertainty in 1/T₁ of ±2%. The NMRD profiles were measured using 1-2 mM solutions of the complexes.

Variable-Temperature ¹⁷O NMR Measurements

The variable-temperature ¹⁷O NMR measurements were recorded on a JEOL EX-90 (2.1 T) spectrometer that was equipped with a 5 mm probe, by using a D₂O external lock. The experimental settings were: spectral width of 10000 Hz, pulse width of 7 μs, acquisition time of 10 ms, 1000 scans, and no sample spinning. Solutions containing 2.6% of ¹⁷O isotope were used. The observed transverse relaxation rates (R_{2obs}⁰) were calculated from the signal width at half height.

Luminescence Measurements for Inner-Sphere Water Molecules Q

Luminescence lifetime measurements were all obtained on a Perkin-Elmer LS 50B spectrofluorimeter with a 420 nm cut-off filter. The concentration of the complex was 1×10⁻⁵˜1×10⁻⁴ mol dm⁻³. The emission of Tb³⁺ or EU³⁺ complexes was monitored at 615 nm and 545 nm, respectively. Gd³⁺ is a neighbor of Eu³⁺ and Tb³⁺ with a nearly identical ionic radius, and thus their chemistries are virtually identical. Therefore, the Horrocks equation for determining the number of coordinated water molecules q for Eu³⁺ and Tb³⁺ is still valid for the calculation of the q value of Gd³⁺. In general, the number of coordinated water molecules q can be determined by the measurement of the luminescent lifetime of the complexes separately in H₂O and D₂O by application of the Horrocks equation.

\dot{q}_{corr}=\dot{A}(k_{H}_{₂}_{O}−k_{D}_{₂}_{O}−a)

where k is the constant for depopulation (reciprocal of luminescent lifetime), and A is a proportionality constant that signifies the sensitivity of the lanthanide ion to vibronic quenching by OH oscillators. a is correlation value for the consideration of the outer-sphere water molecules.

In Vivo MRI Measurements.

To study the MRI contrast and biodistribution behavior of gadolinium complexes GdL1-GdL3 with pendant crown ethers, experiments were performed using male Sprague-Dawley (SD) rats that weighted 150-180 g, and were housed at the Wuhan Institute of Physical and Mathematics of the Chinese Academy of Sciences (Wuhan, Hubei Province, China). All procedures conformed to the institutional guidelines for animal welfare. The rat was injected via the femoral vein with 1 mL saline solution of GdL1 in concentrations of 0.1, 0.033, and 0.02 mM/kg, which are the standard dosage, one third and one fifth of the standard dosage used in clinic currently. MR imaging was performed on a 4.7 T Bruker BIO-SPEC-47/30 MRI scanner with a custom spiral surface coil. The animal was scanned in the prone position in the MRI experiment. Prior to the administration of MRI contrast agents, coronal flow-compensated T₁-weighted fast spin-echo images were acquired by multi-slice and multi-echo (MSME) techniques using TR=300 ms, TE=15 ms, four averages, field of view (FOV) 5×5 cm² or 12×5 cm², and a matrix of 128×256. Immediately during and after the injection of the CAs, a dynamic fast spin-echo sequence was used to acquire T₁-weighted images from six 2 mm sections (with 1.5 mm gaps, TR=300 ms, TE=14 ms, four averages, FOV 5×5 cm² or 12×5 cm², and a matrix of 128×256). The total duration of the dynamic scanning lasted up to 400 min after the administration, with an image every 4 min. A water tube was placed in the field of view as a phantom reference. The intensity enhancement (IE) of the region of interest (RI) at time point t is expressed by

IE=100% (RI₍ₜ₎−RI₍₀₎)/RI₍₀₎

where RI₍ₜ₎ corresponds to the normalized signal intensity as measured at time point, t and RI₍₀₎ is the normalized signal intensity precontrast.

In Vitro Cytotoxicity Measurements

To evaluate the toxicity of prepared Gd³⁺ complexes, a colorimetric assay (XTT-based) for the non-radioactive quantification of cell proliferation was applied. In the cell proliferating process, the added tetrazolium salt XTT was cleaved to a formazan dye by metabolically active cells, and the quantification of cell growth was achieved by the variation of the absorbance at 492 nm. Seed MIHA Cells at a concentration of 5×10³ cells/well (tissue culture grade, 96 wells, flat bottom) were added to Gd³⁺ complexes (0.625-10 mM) in a humidified atmosphere (37° C., 6.5% CO₂). Different concentrations of drugs were adjusted by performing serial dilution. The incubation period of the cell culture ranged from 1, 2, 8, and 24 hrs. XTT dye at a concentration of 5 mg/mL (sigma, St. Louis, Mo.) was added, and the plates were incubated for 12 h in a moist chamber at 37° C. The optical density was determined by eluting the dye with dimethyl sulfoxide, and the absorbance was measured at 570 nm. At least three independent experiments were performed.

In view of the many possible embodiments to which the principles of our inventions may be applied, it should be recognized that the illustrated embodiments are only examples of the invention, and should not be taken as a limitation to the scope of the invention. Rather, the scope of the invention is defined by the following claims. We therefore claim as our invention, all that comes within the scope and spirit of these claims.

We claim:

1. A magnetic resonance imaging contrast agent with prolonged residence time in vivo, which comprises a pendant crown ether compound covalently bound to a metal chelator that contains a paramagnetic ion in physiologically acceptable diluent, the pendant crown ether having the structure: 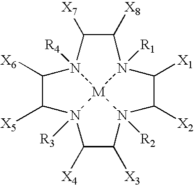
wherein, M is a paramagnetic metal ion;
wherein, only one of R1-R4 comprises a pendant crown compound, and the other three R groups are the chelating groups —CH₂COO—, —CH₂COO—, —CH₂CONH—, —CH₂COC—, —CH₂OH, or —CH₂O—;
wherein, the metal chelators comprise 1,4,7,10-tetra-(acetic acid)-1,4,7,10-tetraazacyclododecane (DOTA), 1,4,7-tris-(acetic acid)-1,4,7,10-tetraazacyclododecane (DO3A), 1-(2-hydroxypropyl)-4,7,10-tris-(acetic acid)-1,4,7,10-tetraazacyclododecane (HP-DO3A), 1-(1-(hydroxymethyl)-2,3-dihydroxypropyl)-1,4,7-tris(carboxylmethyl)-1,4,7,10-tetraazacyclododecane (DO3A-butrol);
wherein, X₁-X₈ each comprises a hydrogen, alkyl, aryl, alcohol, amine, amido, nitro, ether, ester, ketone, imino, aldehyde, alkoxy, carbonyl, halogen, sulfur containing moieties, phosphorous containing moieties, silicon containing moieties, or other blocking moieties, or together with an adjacent X group is an alkyl or aryl group; and
wherein, there are at least one and at most two water molecules that are coordinated with the paramagnetic metal ion directly.

2. The magnetic resonance contrast agent of claim 1, wherein the compound is an MRI contrast agent and the paramagnetic metal ion is chromium (III), manganese (II), iron (II), iron (II), cobalt (II), nickel (II), copper (II), praseodymium (III), neodymium (III), samarium (III), gadolinium (III), terbium (III), dysprosium (III), holmium (III), erbium (III), ytterbium (III), or a combination thereof.

3. The magnetic resonance contrast agent of claim 2 wherein the paramagnetic metal ion is gadolinium (III).

4. The magnetic resonance contrast agent of claim 1, wherein the compound is an MRI contrast agent and the macrocyclic chelator is 1,4,7-tris-(acetic acid)-1,4,7,10-tetraazacyclododecane (DO3A).

5. The magnetic resonance contrast agent of claim 1, wherein the compound is an MRI contrast agent and the pendant crown compound is a crown ether.

6. The magnetic resonance contrast agent of claim 5, wherein the pendant crown ether is an oxa crown ether, an aza crown ether, a thia crown ether, an aza-oxa crown ether, an oxa-thia crown ether, an aza-thia crown ether, an aromatic crown ether, or a derivative thereof.

7. The magnetic resonance contrast agent of claim 6, wherein the pendant crown ether is aza-15-crown-5, a 15-crown-5, or a quinine alkylated diaza-18-crown-6.

8. The magnetic resonance contrast agent of claim 1, wherein the pendant crown compound is connected to a macrocyclic chelator with an alkyl, allyl, alkyne, aryl, amide, ester, ether, ketone, imino group, phosphorous containing moieties, sulfur containing moieties, silicon containing moieties, ethylene glycol, polyethylene glycol, peptide, or polypeptide.

9. A method for kidney imaging, which comprises administering the magnetic resonance contrast agent of claim 1 to a subject, permitting the agent to accumulate at a site of the renal cortex and medulla of a kidney for which an image is desired, performing an MRI scan of the site, and generating an image therefrom.

10. A method for kidney imaging, which comprises-administering the magnetic resonance contrast agent of claim 2 to a subject, permitting the agent to accumulate at a site of the renal cortex and medulla of a kidney for which an image is desired performing an MRI scan of the site, and generating an image therefrom.

11. The method of claim 10, wherein the magnetic resonance contrast agent shows prolonged residence in the kidney.

12. The method of claim 11, wherein the magnetic resonance contrast agent is administered at a dosage of 0.02 mmol/kg, 0.033 mmol/kg, or 0.1 mmol/kg.

13. The method of claim 11 wherein renal intensity enhancement reaches its maximum within a few minutes after the administration of the contrast agent and renal MR signal enhancement lasts for at least 200 minutes in vivo.

14. A method for liver imaging, which comprises administering the magnetic resonance contrast agent of claim 1 to a subject, permitting the agent to accumulate at a site of hepatic parenchyma in a liver for which an image is desired, performing an MRI scan of the site, and generating an image therefrom.

15. The method of claim 14, wherein the magnetic resonance contrast agent shows prolonged residence in the liver.

16. The method of claim 14, wherein the magnetic resonance contrast agent is administered at a dosage of 0.02 mmol/kg, 0.033 mmol/kg, or 0.1 mmol/kg.

17. The method of claim 14, wherein hepatic intensity enhancement reaches its maximum about 80-100 minutes after administering the contrast agent and hepatic MR signal enhancement lasts for at least 250 minutes in vivo.
